(19) United States
(12) Patent Application Publication
(10) Pub. No.: US 2024/0161932 A1
(43) Pub. Date: May 16, 2024

(54) SYSTEMS FOR ASSESSING RISK OF DEVELOPING BREAST CANCER AND RELATED METHODS

(71) Applicant: Gabbi, Inc., Portland, OR (US)

(72) Inventors: Kaitlin Christine, Portland, OR (US); Ashmitha RAJENDRAN, Portland, OR (US); Balaji KESAVAN, Beaverton, OR (US)

(21) Appl. No.: 18/476,014

(22) Filed: Sep. 27, 2023

Related U.S. Application Data
(63) Continuation of application No. PCT/US2022/022216, filed on Mar. 28, 2022.
(60) Provisional application No. 63/167,119, filed on Mar. 28, 2021.

(51) Int. Cl. G16H 50/30; G16H 50/70

(52) U.S. Cl. CPC G16H 50/30; G16H 50/70

(57) ABSTRACT

Systems and methods for training a machine learning model to assess the risk of a human subject for developing a disorder are described. An exemplary method includes receiving an input dataset corresponding to human subjects, splitting the input dataset into a first dataset corresponding to a first portion of the human subjects and a second dataset corresponding to a second portion of the human subjects, selecting a risk factor associated with developing the disorder from the first dataset, training a machine learning model using the first dataset and the risk factor, providing the second dataset to the machine learning model to generate a risk prediction for developing the disorder for each human subject in the second portion, and tuning at least one parameter of the machine learning model based on the generated risk predictions.

### CROSS REFERENCE

This application is a continuation application of International Patent Application No. PCT/US2022/022216, filed on Mar. 28, 2022, which claims the benefit of U.S. Provisional Patent Application No. 63/167,119, filed Mar. 28, 2021, each of which is incorporated by reference herein in its entirety.

### TECHNICAL FIELD

The present disclosure relates to methods and systems for assessing the risk of a subject (e.g., human male or human female patients referred to as “users” throughout this disclosure) for developing a disorder, a disease (e.g., cancer), medical condition, and/or syndrome using machine learning, and more particularly, to digital health software designed to improve accessibility and interpretability of breast cancer risk assessment and early detection measures for female patients (defined as people who are assigned female at birth) based on machine learning algorithms.

### BACKGROUND

About one in eight women in the United States will develop an invasive form of breast cancer. Additionally, breast cancer is the second deadliest cancer behind lung cancer. Today, every year in the United States, 38,000 women of all ages and ethnicities die from breast cancer due to delayed diagnosis. Not only does this cost precious lives, but it's also an incredible burden on the system. Every year, health insurers (or “payors”) pay $163 billion due to late diagnoses and the associated healthcare costs.

Furthermore, knowledge of health and diseases affects retention of health and medical plans and sentiment towards the medical community. Currently, there are an overwhelming number of sources available with health and medical literature; however, trust and comfortability drives action and engagement in acquiring health and medical knowledge. Studies have shown that there is a lack of trust in the medical system and women consult their mothers, sisters, and friends before reaching out to a medical professional.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY

At least one aspect of the present disclosure is directed to a computer-implemented method for training a machine learning (ML) model to assess the risk of a human subject for developing at least one disorder. The method includes receiving an input dataset including at least medical claim data corresponding to a plurality of human subjects over a target prediction period, splitting the input dataset into a first dataset corresponding to a first portion of the plurality of human subjects and a second dataset corresponding to a second portion of the plurality of human subjects, selecting at least one risk factor associated with developing the at least one disorder from the first dataset, training a machine learning (ML) model using the first dataset and the at least one risk factor, the ML model including at least one logistic regression model, providing the second dataset to the ML model to generate a risk prediction for developing the at least one disorder by the end of the target prediction period for each human subject included in the second portion of the plurality of human subjects, and tuning at least one parameter of the ML model based on the generated risk predictions for the second portion of the plurality of human subjects.

In some embodiments, the first dataset is a training dataset and the second dataset is a validation dataset. In some embodiments, the method includes creating a third dataset corresponding to a third portion of the plurality of human subjects, and providing the third dataset to the ML model with the at least one adjusted parameter to generate a risk prediction for developing the at least one disorder by the end of the target prediction period for each human subject included in the third portion of the plurality of human subjects. In some embodiments, the method includes determining whether each human subject of the plurality of human subjects has developed the at least one disorder by the end of the target prediction period, labeling a portion of the plurality of human subjects who have developed the at least one disorder by the end of the target prediction period as positive for the disorder, and labeling a remaining portion of the plurality of human subjects as healthy.

In some embodiments, determining that a human subject has developed the at least one disorder includes detecting at least one identifying factor in a final year of the target prediction period. In some embodiments, the first portion of the plurality of human subjects has a first ratio of positive to healthy human subjects and the second portion of the plurality of human subjects has a second ratio of positive to healthy human subjects. In some embodiments, the first ratio and the second ratio are different. In some embodiments, selecting the at least one risk factor associated with developing the at least one disorder includes identifying at least one risk factor in a first year of the target prediction period associated with a diagnosis of the at least one disorder by the end of the target time period.

In some embodiments, the at least one risk factor corresponds to at least one Clinical Classifications Software Refined (CCSR) category. In some embodiments, the at least one disorder is breast cancer. In some embodiments, the trained ML model is configured to receive input data corresponding to a user and provide a risk prediction indicating the user's risk of being diagnosed with the at least one disorder by the end of the target prediction period. In some embodiments, the risk prediction includes a risk score.

Another aspect of the present disclosure is directed to a system for training a machine learning (ML) model to assess the risk of a human subject for developing at least one disorder. The system includes one or more computer systems programmed to perform operations comprising: receiving an input dataset including at least medical claim data corresponding to a plurality of human subjects over a target prediction period, splitting the input dataset into a first dataset corresponding to a first portion of the plurality of human subjects and a second dataset corresponding to a second portion of the plurality of human subjects, selecting at least one risk factor associated with developing the at least one disorder from the first dataset, training a machine learning (ML) model using the first dataset and the at least one risk factor, the ML model including at least one logistic regression model, providing the second dataset to the ML model to generate a risk prediction for developing the at least one disorder by the end of the target prediction period for each human subject included in the second portion of the plurality of human subjects, and tuning at least one parameter of the ML model based on the generated risk predictions for the second portion of the plurality of human subjects.

In some embodiments, the one or more computer systems is programmed to perform operations comprising: creating a third dataset corresponding to a third portion of the plurality of human subjects, and providing the third dataset to the ML model with the at least one adjusted parameter to generate a risk prediction for developing the at least one disorder by the end of the target prediction period for each human subject included in the third portion of the plurality of human subjects. In some embodiments, the one or more computer systems is programmed to perform operations comprising: determining whether each human subject of the plurality of human subjects has developed the at least one disorder by the end of the target prediction period, labeling a portion of the plurality of human subjects who have developed the at least one disorder by the end of the target prediction period as positive for the disorder, and labeling a remaining portion of the plurality of human subjects as healthy.

In some embodiments, determining that a human subject has developed the at least one disorder includes detecting at least one identifying factor in a final year of the target prediction period. In some embodiments, the first portion of the plurality of human subjects has a first ratio of positive to healthy human subjects and the second portion of the plurality of human subjects has a second ratio of positive to healthy human subjects. In some embodiments, selecting the at least one risk factor associated with developing the at least one disorder includes identifying at least one risk factor in a first year of the target prediction period associated with a diagnosis of the at least one disorder by the end of the target prediction period.

In some embodiments, the at least one risk factor corresponds to at least one Clinical Classifications Software Refined (CCSR) category. In some embodiments, the at least one disorder is breast cancer. In some embodiments, the trained ML model is configured to receive input data corresponding to a user and provide a risk prediction indicating the user's risk of being diagnosed with the at least one disorder by the end of the target prediction period. In some embodiments, the risk prediction includes a risk score.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from the foregoing and the following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 is a block diagram of a platform in accordance with aspects described herein;

FIG. 2 is a flow diagram of a method for building and training a risk assessment model in accordance with aspects described herein;

FIGS. 3A-3C illustrate example input parameters and characteristics in accordance with aspects described herein;

FIG. 4 illustrates several example hypothesized risk factors in accordance with aspects described herein;

FIG. 5 illustrates several example cohorts in accordance with aspects described herein;

FIG. 6 illustrates several example plots of log odds for various risk factors in accordance with aspects described herein;

FIG. 7 illustrates several example plots of log odds for various transformed risk factors in accordance with aspects described herein;

FIG. 8A is a flow diagram of a method for building and training a risk assessment model in accordance with aspects described herein;

FIG. 8B is a flow diagram of a method for building and training a risk assessment model in accordance with aspects described herein;

FIG. 9A is a flow diagram of a method for building and training a risk assessment model in accordance with aspects described herein;

FIG. 9B is a flow diagram of a method for building and training a risk assessment model in accordance with aspects described herein; and

FIG. 10 illustrates an example computing device.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

Systems and methods for providing a comprehensive data analytics platform that virtually assesses a user's risk of developing a disease (e.g., cancer) is described herein. In at least one embodiment, clinical risk assessments and genetic risk assessments are combined to improve risk analysis of a particular disease (e.g., cancer), medical condition, and/or syndrome. In some examples, machine learning models and algorithms are utilized to determine such a risk assessment of a user (e.g., human female or human male).

Risk assessment models currently available attempt to solve the aforementioned problems; however, often times, these existing risk assessment models fall short of expectations with respect to optimal risk assessment. For example, models such as the BOADICEA, Tyer-Cuzick, Gail, and/or BRCAPRO models can determine the initial risks of a woman by regression-based assessments, which are considered standard of care and are present in the medical guidelines. These risk assessment tools, however, are developed and trained on limited datasets (e.g., only 100s of subjects at most), and with a severe lack of participant diversity (e.g., focused on Caucasian women in North America and Western Europe). The foregoing leads to the models being skewed and, as a result, only truly benefit those that fit into the limited parameters of the training sets.

If caught at early stages 0 and 1, breast cancer 5-year relative survival rates are approximately 90%. However, a majority of people who are susceptible to breast cancer are unaware of the risks, and, therefore, do not maintain breast health including medical check-up necessities, and monitoring for signs. Therefore, a need exists to address all of these gaps in public awareness.

Additionally, the standard of care for risk assessment is inadequate for diverse populations (e.g., White, Black, Hispanic/Latino, Asian, etc.) and varying demographics (e.g., socioeconomic diversity, communities of color, low income or poverty-stricken communities, etc.). It is known that there are a multitude of risk factors associated with breast cancer development, but conventional assessment tools do not account for all possible ones, quantitate the influence of each one, or associate different factors with each other.

Therefore, a need exists to provide a risk assessment platform that can account for the aforementioned problems by creating a risk assessment modeling tool that accounts for diverse populations and varying demographics.

The systems and methods described in the present disclosure relate to digital health software solutions designed to improve accessibility and interpretability of disease (e.g., breast cancer) risk assessment. In some examples, the solutions provided herein utilize machine learning to achieve improved accessibility and interpretability of disease risk assessment.

The benefits of this approach include non-linearity, whereas existing models and standards of care assume linear relationships between risk factors and breast cancer risk assessment. The systems and methods described herein, according to exemplary embodiment, describe risk assessment model(s) that incorporate the use of specific machine learning techniques within the workflow to quantitatively define the learned relationships in order to remove uncertainty.

In some embodiments, the systems and related methods herein use a central platform that aims to solve the aforementioned problems (i.e., loss of lives due to failed early detection of a disease and the cost burden on the healthcare system of attempting to treat or cure that disease) by saving lives and saving payors' money by providing users (e.g., human males and human females concerned with potentially developing a lethal disease such as breast cancer) with a risk assessment analysis using a wide berth of data together with machine learning.

In some embodiments, the systems and methods described herein provide a community-based approach to holistic disease assessment and management. The platform provided herein, according to an exemplary embodiment, is capable of aggregating meaningful data to provide personalized assessments, tailored action plans, and engaging communities.

Various systems, methods, techniques, processes and flow diagrams will now be described in detail with reference to a few exemplary embodiments (e.g., risk assessment modules) as illustrated in the accompanying drawings. In the following description, numerous specific details are set forth in order to provide a thorough understanding of one or more aspects and/or features described or reference herein. It will be apparent, however, to one skilled in the art, that one or more aspects and/or features described or referenced herein may be practiced without some or all of these specific details. In other instances, well known process steps and/or structures have not been described in detail in order to not obscure some of the aspects and/or features described or reference herein.

### Platform

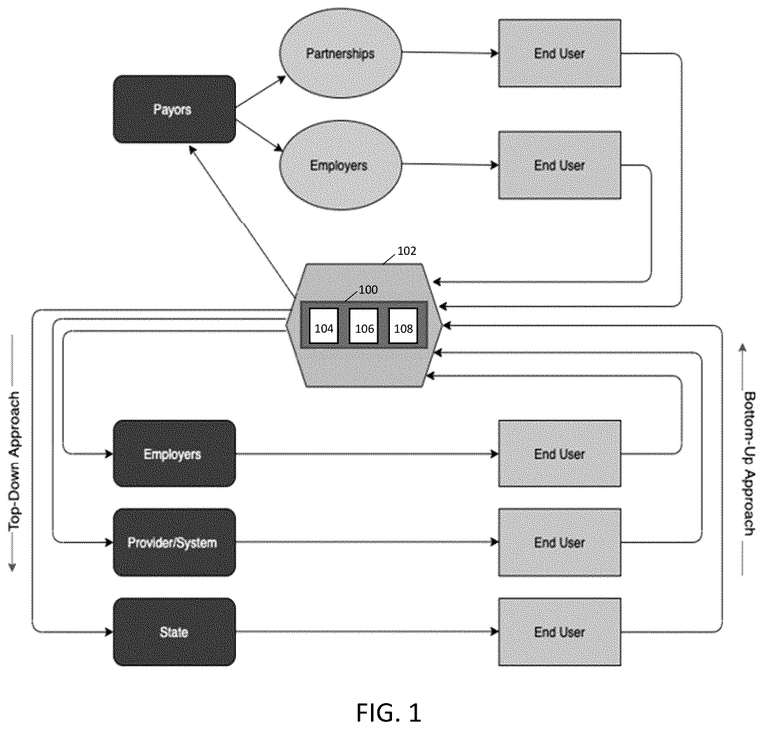

FIG. 1 illustrates a platform 100 in accordance with aspects described herein. In some instances, throughout this disclosure, the platform 100 is referred to by its brand name, “Gabbi.” In an exemplary embodiment, the platform 100 refers to a digital health software designed to improve accessibility and interpretability of breast cancer risk assessment and early detection measures for female patients (defined as people who are assigned female at birth and referred to as users throughout). In some embodiments, the platform 100 is used in conjunction with associated hardware (e.g., servers, storage, processors, etc.) to either store and/or execute the software/algorithms according to exemplary embodiments taught throughout this disclosure. In some embodiments, the platform 100 is implemented on a platform server 102. The platform server 102 comprises software components and databases that are, in some embodiments, deployed at one or more data centers (not shown) in one or more geographic locations, for example.

In an exemplary embodiment, the platform 100 is in electronic communication with devices capable of a user input (e.g., user devices) or having graphical user interfaces (GUIs) for entering data into the platform 100's software. The device include mobile phones, PC computers, tablets, or any other suitable electronic device for inputting a user selection or user data (e.g., data related to a human subject, such as, height, weight, demographic information, socioeconomic information, etc.). In another exemplary embodiment, this data is acquired through insurance companies and/or payors.

The platform 100, according to an exemplary embodiment, presents necessary breast cancer and breast health information digestible for the layperson, and suggests a personalized medical plan. In some embodiments, the personalized medical plan is based on the National Cancer Consortium Network and/or other medical and clinician based literature. Based on zip code, insurance, and personal factors, users (e.g., human male or human female subjects in search of determining their risk assessment for developing a particular disease or disorder) are suggested appropriate physicians and clinics for follow ups.

In some embodiments, the platform 100 utilizes one or more machine learning algorithms (or models) and/or artificial intelligence models. In some embodiments, the machine learning algorithms and/or artificial intelligence models enable the platform 100 to assist the user (e.g., human subject) in determining a more accurate risk assessment of developing a disease, disorder, etc. the machine learning algorithms, in some embodiments, are configured to provide personalization, and adapted to foster improved engagement between users and the platform 100 (e.g., the digital health software taught herein) to improve clinical outcomes (e.g., surviving past their anticipated mortality rate due to a disease or cancer).

In some examples, the platform 100 includes a community engagement engine 104. In this platform 100, users are grouped into cohorts based on risk factors and action plan similarities such as social determinants of health (i.e. geographic location, ethnicity, socio-economic status, etc.), risk level, and family history. The community engagement engine 104 incites cohort engagement with activations and intra-cohort communications with open discussions and activities. Ultimately, the goal of incorporating cohort participation into the user's action plan is to encourage adherence to health plans and treatments, improve attitude towards the medical field, and better prevention, early detection, and improve health outcomes. Encouraging engagement involves understanding which features and topics are most successful at doing so. Using generative statistical models, the community engagement engine 104 analyzes which sentiments are associated with which engagement topics and deduce which topics promote the most interactions.

Knowledge of health and diseases can affect retention of health and medical plans and sentiment towards the medical community. Currently there are an overwhelming number of sources available with health and medical literature, however trust and comfortability drives action and engagement in acquiring health and medical knowledge. In many cases, women consult their mothers, sisters, and friends before reaching out to a medical professional. The platform 100 is configured to combine education and trust to be a familiar resource that women can consult because it is backed by medical expertise and presents as a comfortable and reliable coach.

The platform 100, according to an exemplary embodiment, includes software that is used by the user to be their “best friend,” “sister,” and “mom” with the medical expertise of a personal physician (e.g., oncologist). The platform 100 incorporates personalization and precision based action plans and presentation by taking in personal determinants of health, aggregating them in a meaningful way, to provide unique insights.

In some embodiments, the platform 100 includes a risk assessment engine 106 using an interpretable machine learning model that quantifies relationships between patient characteristics, demographics, and other information from medical claims and breast cancer risk. The risk assessment engine 106 includes a risk assessment model, which is referred throughout this disclosure as the Gabbi Risk Assessment Model or “GRAM.”

In some embodiments, the risk assessment engine 106 is configured to develop the risk assessment model using interpretable machine learning using all factors available from medical claims datasets. For example, in some embodiments machine learning-based clustering and feature selection identifies most impactful clinical factors for breast cancer prediction. The risk assessment engine 106 is used to significantly enhance clinical understanding of influential risk factors and/or improve risk prediction.

In some embodiments, the platform 100 includes a community action engine 108. In some examples, the community action engine 108 is configured to provide a “Gabbi Action Plan” (GAP) and/or a “Gabbi Community”, which provides personalized clinical suggestions and create an encouraging community. In doing so, the platform 100 is used to develop personalized outcome reports from clinically and medically derived recommendations for breast health maintenance and steps personalized for each risk assessment and combination of risk factors and validate with medical and clinical expertise.

In some embodiments, the GAP encourages maintenance of breast health, and early detection of nodules or cancer. The GAP plan is derived from clinical and medical literature that correlate risk and general health and life stages to clinically driven plans (e.g., the National Comprehensive Cancer Network, National Society of Genetic Counselors, United States and Preventative Task Force, and/or clinicians associated with the platform 100).

With the GAP, users are given a notification of their next actionable item. In some embodiments, this also includes notifications when their engagement cohorts are in communication or active. As users complete tasks or communicate in the engagement platform, they are asked to “mark” their completion off (e.g., like a task list).

On the user application (e.g., an app installed on the user's phone), the medical tasks (e.g., self-breast exams, mammograms, set up general medical checkup appointments) are clearly defined with user friendly instructions and reasonings. It should be appreciated that the medical tasks are recommendations and that official medical evaluations are performed by the user's clinician (resources for one will be provided per user on their action plan based on zip code, insurance, and physician preferences).

In some embodiments, the platform 100 is configured to provide an engagement sub-platform, which is a subset of the overall platform 100. In some embodiments, the engagement sub-platform significantly increases engagement resulting in health outcome improvement. According to an exemplary embodiment, the GRAM workflow groups subjects (e.g., women) into specific groups, cohorts, based on the GRAM results (specifically grouping women in cohorts who have similar risk levels). Women are grouped based on similar risks and put into curated cohorts. In some examples, similarities are determined by comparing top risk factors. In one exemplary embodiment of the platform 100, non-cohort members cannot join and be part of the internal cohort conversations, however there are a variety of “open groups” based on popular topics of discussion found within the cohort (i.e., breast health, mammograms, biopsies, family history).

The social component of the platform 100, much like a dating application or networking application, is configured to send notifications that prompt joining conversations, and notifications around new topics or questions of discussion within cohort and groups. Each individual female member creates a profile, similar to a resume or CV, but with pertinent personal information and health related data such as risk factors, family history, diagnosis. Members initiate conversations, participate in group conversations, and share their action plans as they make progress together. It is appreciated that “female” subject is referred to as the main user or subject using the platform 100, the platform 100 is not limited to human females, but may also be used for risk assessment of human males.

Within the GAP, tracking is conducted in conjunction with IoT devices (e.g., smart watches, fitness trackers, etc.) in regard to the steps that are taken, when, where, the length of time that it takes, etc. A member is able to self-select which path he or she wants to take based on difficulty: minimal/small interventions like lifestyle changes, medium interventions like diagnostic exams, and large interventions like surgical interventions. Notifications are connected to the user's device (e.g., smartphone, smart watch, etc.) to alert and notify the member on community discussions, new questions and conversations, when to take the next step on the action plan and when they have “unlocked” risk reduction over time.

In some examples, the platform 100 is configured to determine ease of use of the platform 100 by using quantitative metrics to guide user testing. For example, in some embodiments quantitative success is derived from search engine analytics (e.g., Google) by measuring threshold of engagement, short- and long-term retention, screen flow, and identifying which features, topics, and content are most interacted. In some embodiments, an alpha version of the platform 100 (or a respective application) is released to a consumer base representative of the platform 100's target market (e.g., female at birth, ages 35+, and access to a phone or computer). For example, in some embodiments this initial release is performed with at least 150 participants. The amount of success is be determined by frequency and amount of active user interaction with the Gabbi engagement platform and success with GAP completion. The number of guided engagement tasks and prompts per week is adjusted to maintain a specific percentage (e.g., 40% or about 40%) of users interacting and a specific percentage (e.g., 50% or about 50%) of users adhering to medical plans. These metrics indicate the ease of use of each feature, identifying where qualitative tests could indicate improvement tactics.

In some examples, the platform 100 is optimized to improve user engagement, retention, and/or ease of use. For example, in some embodiments after the first 3 months (or about 3 months), the platform 100 determines user perception of the platform and experience (e.g., emotional reactions and ease of use) through usability interviews. Based on quantitative metrics (as described earlier), a group of users (e.g., 8-12 users) are selected based on ease of use, amount of engagement, and personal factors to identify user satisfaction of each feature and optimization opportunities. The group of users are selected based on varying analytical experiences, assessment results, and demographics for usability interviews. These interviews include task-based activities (“Can you calculate your risk?”, “Can you find the interaction platform?”, etc.) and expectation and real time walk-through feedback. In some examples, these tests are repeated (e.g., every 3 months) to optimize the platform 100.

Based on the quantitative metrics and results from the usability research reports, the platform 100 improves user experience and launch a beta version to a larger consumer base (e.g., at least 100 new users). Following the same qualitative and quantitative usability research steps, the platform 100 is configured to gage experience, modify, and release the final product.

In some embodiments the platform 100 is configured to commercialize products by selling to entities such as health insurers (e.g., payors), employers, providers/systems, and/or governments using a “top-down approach.” For example, in some embodiments after proving significance, accuracy, and engagement, the platform 100 is configured to sell to the payors of medium to large organizations. The platform 100 is used to identify each woman specifically who will be getting breast cancer this year and ensure that she engages with necessary preventative strategies at the earliest possible stages when it is most life-saving, treatable, and cheapest for the payor. Payors may want to purchase the platform 100 because the platform 100 significantly decreases their annual spending as it relates to breast cancer delayed diagnosis and treatment today. In the long term, the platform 100 increases member engagement, and increase the lifetime value of the member. In some embodiments, the platform 100 is configured to commercialize products by selling to end users (e.g., individuals) using a “bottom-up approach.” The end users interact with the platform 100 and deliver the platform 100 or associated results (e.g., risk assessment scores) to their employer, provider, payor etc.

### Gabbi Risk Assessment Model (“GRAM”)

As described above, disparities, lack of transparency, and mistrust in medical systems lead to delayed care. Improving attitudes toward the health system and providing personalized, accurate information to patients is imperative. Access to health information drastically improves early diagnosis and outcomes. In some examples, the risk assessment engine 106 of the platform 100 incorporates a demographically inclusive risk assessment tool referred to as the Gabbi Risk Assessment Model (GRAM). In some embodiments, the GRAM utilizes interpretable machine learning methods and non-invasive user information to improve breast cancer risk prediction.

The GRAM provides quantitative insight into breast cancer risk factors where previous studies have had conflicting correlations—including menstrual disorders, other breast conditions, and family histories of cancer. With additional validation through other datasets and biological validation, the GRAM is used as an alternative to standard of care in order to provide the most accurate assessment for patients of all ages and ethnicities.

In some embodiments, developing and training the GRAM includes four phases: member selection, feature selection, training, and prediction. In the member selection phase, eligible members were selected to create an input dataset. The input data is structured with all of the diagnosis information, pharmacy information, and lab information for each member and split into training datasets (e.g., used for the model to learn from) and validation datasets (e.g., to test the model and validate the prediction accuracies). The feature selection phase includes identifying the most influential input risk factors in predicting breast cancer diagnosis using automated methods that return the optimal number and combination of factors. In some examples, the feature selection phase includes eliminating factors that are correlated with each other and provide redundant information (e.g., multicollinearity). In the training phase, the model is trained with the optimal factors retained from the feature selection phase. The model learns the subtleties in the input dataset to derive weights and coefficients per feature, resulting in a quantitative prediction of probability of breast cancer at the end of a target timeframe. During this step, the parameters of the model are fine-tuned using at least one first validation dataset. In the prediction phase, at least one second validation dataset is used to calculate the final accuracy metrics.

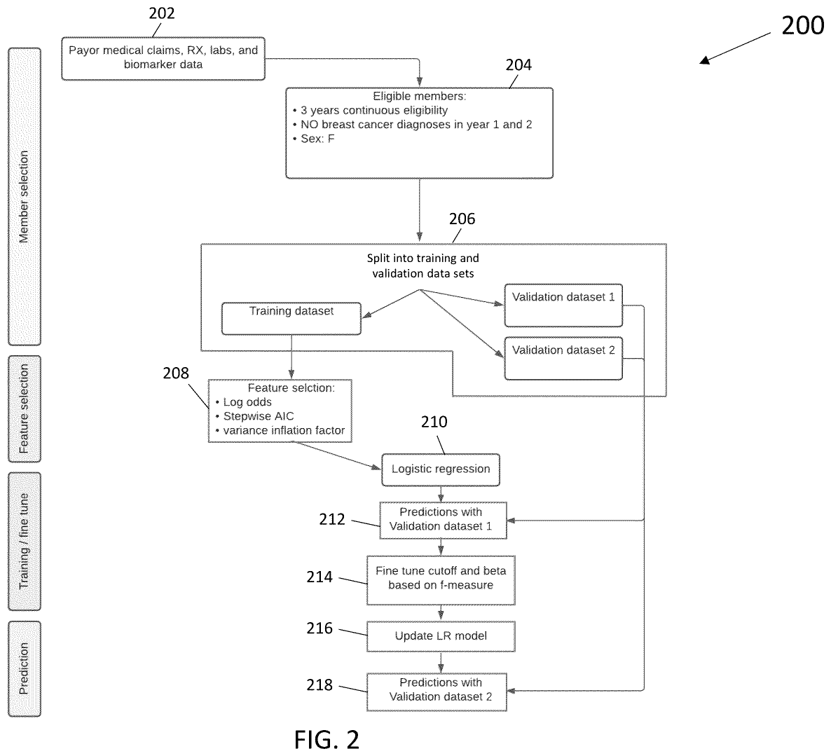

FIG. 2 illustrates a method 200 for developing and training a risk assessment model in accordance with aspects described herein. In some embodiments, the method 200 corresponds to the development and training of the GRAM associated with the platform 100. In some examples, at least a portion of the method 200 is performed by the risk assessment engine 106 of the platform 106.

At step 202, a medical claims dataset is received. In some embodiments, the medical claims dataset includes medical claims (e.g., diagnostic information), pharmacy information, biomarker (e.g., genetic testing done on certain members), and/or lab information associated with a plurality of different individuals (e.g., patients). In some examples, the plurality of individuals represented in the dataset are members of one or more health insurers.

The dataset corresponds to a large number of members (e.g., 5 million, 10 million, 100 million, etc.) and includes data collected over different periods of time (e.g., 6 months, 1 year, 5 years, etc.). Given the large size of the dataset, the dataset is aggregated and/or summarized prior to use for model development. For example, in some embodiments the dataset is organized in groups of diagnoses, labs, and/or pharmacy data and narrowed to members of representative cohorts. Several example entries (or portions of entries) from the medical claim dataset are shown in Table 1 below:

TABLE 1

| Entry | ICD | | CCSR | |
|---|---|---|---|---|
| # | Code | ICD Code Description | Category | CCSR Category Description |
| 1 | C50011 | Malignant neoplasm of nipple and | NEO030 | Breast cancer - all other |
| | | areola, right female breast | | types |
| 2 | C50012 | Malignant neoplasm of nipple and | NEO030 | Breast cancer - all other |
| | | areola, left female breast | | types |
| 3 | N63 | Unspecified lump in breast | GEN017 | Nonmalignant breast |
| | | | | conditions |
| 4 | N630 | Unspecified lump in unspecified | GEN017 | Nonmalignant breast |
| | | breast | | conditions |
| 5 | D0510 | Intraductal carcinoma in situ of | NEO029 | Breast cancer - ductal |
| | | unspecified breast | | carcinoma in situ (DCIS) |
| 6 | D0511 | Intraductal carcinoma in situ of | NEO029 | Breast cancer - ductal |
| | | right breast | | carcinoma in situ (DCIS) |

As shown in Table 1, each entry has a corresponding International Classification of Diseases (ICD) code representing the diagnosis, disease, or injury associated with the claim entry. In some embodiments, the ICD codes are be grouped in Clinical Classifications Software Refined (CCSRs) categories, which are established overarching diagnostic groups where each group encompasses tens to hundreds of similar ICD codes. For example, in some embodiments the ICD codes corresponding to entries #1 and #2 (C50011, C50012) are both included in the same CCSR group (NEO030). Likewise, the ICD code associated with entry #3 (N63) and the ICD code associated with entry #5 (D0510) are included in different CCSR groups (GEN017, NEO029). By organizing the claim entries in CCSR groups, management of the medical claim dataset is improved. For example, in one embodiment a medical claim dataset having over 50,000 ICD codes is reduced to approximately 500 unique CCSR groups.

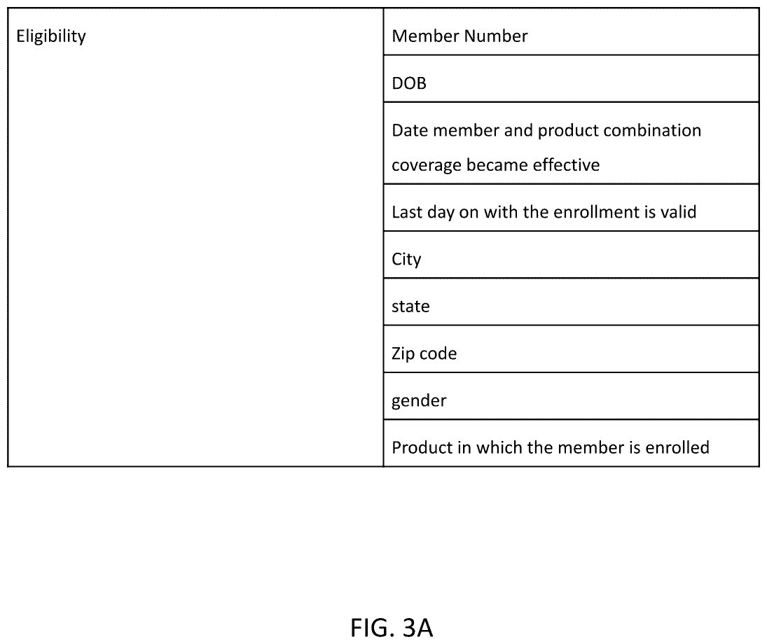

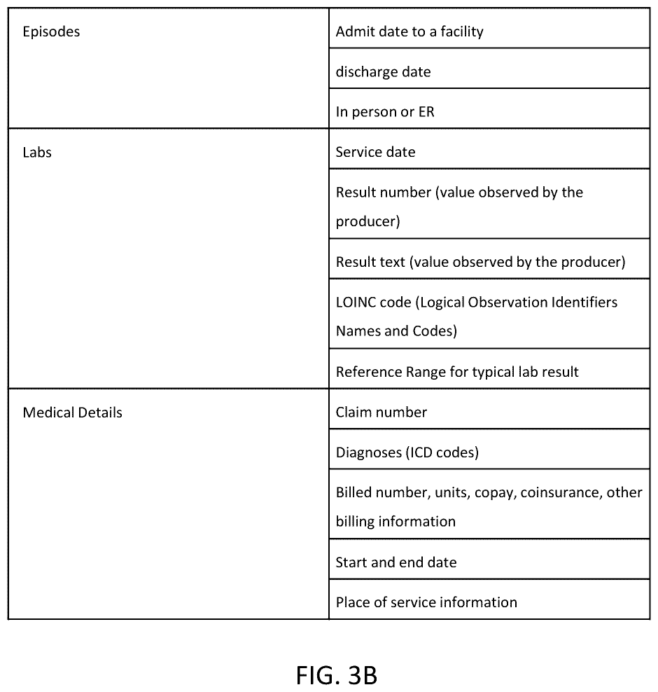

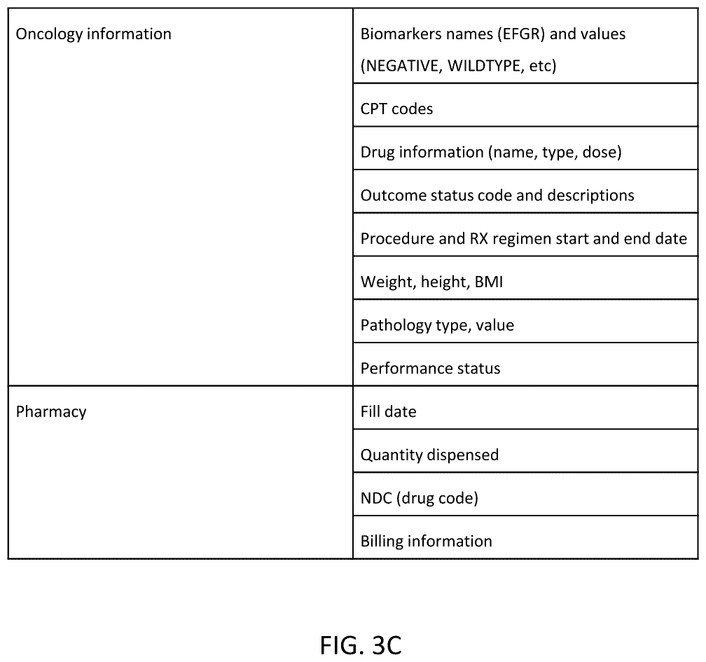

In some embodiments, in addition to medical claims, the input dataset includes other parameters and user characteristics. In some examples, these parameters and characteristics are provided with the medical claim data and/or derived from the medical claim data. In other examples, the parameters are collected from users directly (e.g., a survey or interface provided via a user application in communication with the platform 100). Several examples of such parameters and characteristics are shown in FIGS. 3A-3C.

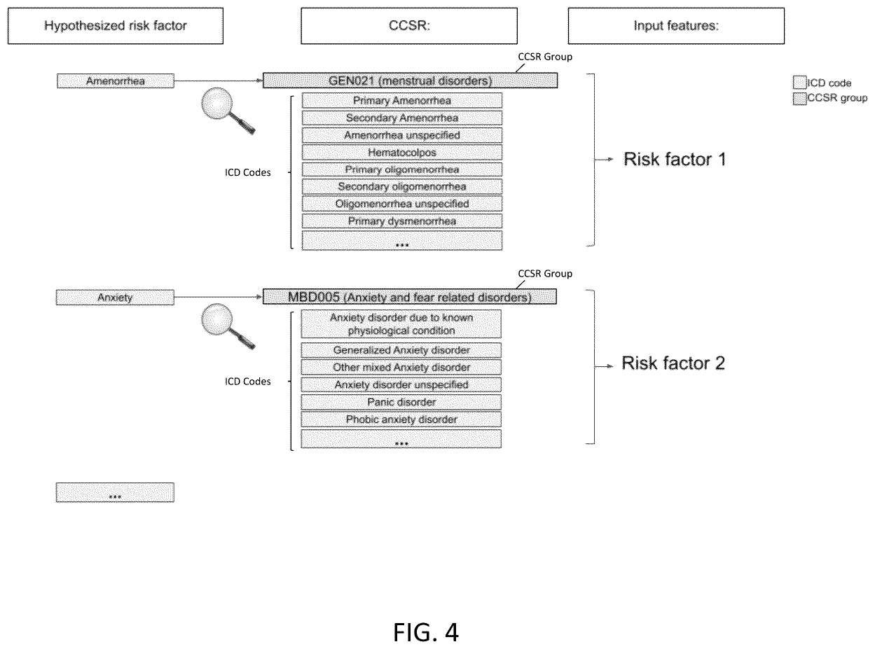

In some examples, risk factors (or hypothesized risk factors) are assigned to different CCSR groups. The hypothesized risk factors correspond to factors, symptoms, or conditions that are believed, suspected, and/or proven to be related to breast cancer diagnoses. FIG. 4 illustrates two example hypothesized risk factors: amenorrhea and anxiety. As shown, the first hypothesized risk factor “amenorrhea” is linked to a first CCSR group GEN021 (menstrual disorders). As such, all medical claim entries having ICD codes included in the first CCSR group are classified under “Risk Factor 1” (i.e., amenorrhea). Likewise, the second hypothesized risk factor “anxiety” is linked to a second CCSR group MBD005 (anxiety and fear related disorders). As such, all medical claim entries having ICD codes included in the second CCSR group are classified under “Risk Factor 2” (i.e., anxiety). In other examples, specific ICD codes are used to identify CCSR groups that are relevant to breast cancer diagnosis (e.g., to be used as risk factors).

While the example above describes two different risk factors, it should be appreciated that many risk factors are considered in some embodiments (e.g., dozens, hundreds, thousands, etc.). In some examples, the types of risk factors used for model development are narrowed based on biological validity. In certain examples, the biological validity of different risk factors is determined by a board of practicing medical professionals. For example, in some embodiments endometriosis is considered as a risk factor based on evidence of environmental and molecular links with breast cancer. Likewise, conditions that involved aberrant hormone production including polycystic ovarian syndrome (PCOS); abnormal uterine bleeding (menorrhagia, metrorrhagia, anovulation); metabolic and endocrine related syndromes; gastrointestinal disorders and syndromes; anxiety, depression and related disorders; complications during or post-pregnancy and any personal or familial histories of said conditions are used or considered as risk factors for model development. In some examples, given that endometrial hyperplasia, endometrial cancer, and ovarian cancer have been associated with specific genetic mutations that also increase the risk of breast cancer, these conditions are used or considered as risk factors. In other examples, conditions, diseases, disorders, symptoms, or any other attribute are used or considered as risk factors without medical evaluation.

At step 204, eligible members are identified from the medical claims dataset. In some embodiments, the eligibility of each member is determined based on medical claim data over a target prediction period. For example, in some embodiments an n-year prediction period corresponds to n+1 years of medical claim data (e.g., n years and a prediction year). In some examples, a two-year prediction period is used to develop the model. A compelling reason for selecting a two-year timeframe is that mammography can detect the presence of a lump two years before they can be felt externally. Given this, identifying high risk patients prior to any palpable mass allows for potential clinical action to minimize the burden of disease. Additionally, on average, employees stay with a single employer for two to five years. Given that the employer provides medical insurance, employees typically have medical coverage for this length of time, and medical claim data can be collected over at least this time period.

In some embodiments, a member is determined as eligible if the medical claim data associated with said member does not include any breast cancer related claims prior to the last year of the prediction period. For example, in some embodiments a member is determined as eligible for a two-year prediction period if the member's medical claim data does not include any breast cancer related claims in the first and second years. In certain examples, eligibility is determined based on additional criteria (e.g., female gender).

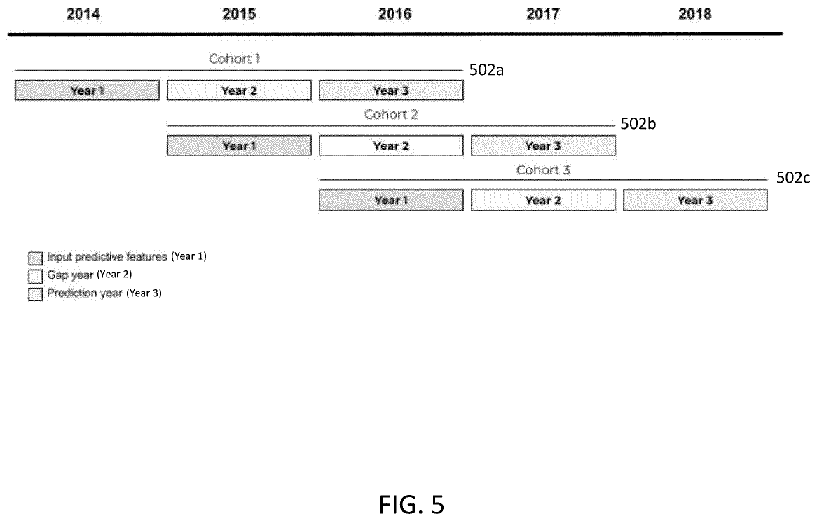

The target prediction period is used to define multiple cohorts of eligible members. FIG. 5 illustrates an example plurality of cohorts 502 distributed across a five year time period. In some embodiments, each cohort of the plurality of cohorts 502 corresponds to a two-year prediction period (e.g., three years of medical claim data). Each cohort includes eligible members with continuous coverage across three years. For example, in some embodiments the first cohort 502a includes eligible members having medical claim coverage from 2014 to 2016. Predictive features of the first cohort 502a members are identified in year 1 (e.g., Jan. 1, 2014 to Dec. 31, 2014) to predict whether a breast cancer diagnosis would be given in year 3 (e.g., Jan. 1, 2016-Dec. 31, 2016). The second cohort 502b includes eligible members having medical claim coverage from 2015 to 2017. Predictive features of the second cohort 502b members are be identified in year 1 (e.g., Jan. 1, 2015 to Dec. 31, 2015) to predict whether a breast cancer diagnosis would be given in year 3 (e.g., Jan. 1, 2017-Dec. 31, 2017). The third cohort 502c includes eligible members having medical claim coverage from 2015 to 2017. Predictive features of the third cohort 402c members are be identified in year 1 (e.g., Jan. 1, 2016 to Dec. 31, 2016) to predict whether a breast cancer diagnosis would be given in year 3 (e.g., Jan. 1, 2018-Dec. 31, 2018).

In some embodiments, the breast cancer diagnosis of eligible members (e.g., members in the plurality of cohorts 502) is determined based on the presence of one or more ICD codes in a member's medical claim data. For example, in some embodiments a breast cancer diagnosis is defined using codes in the NEO030 CCSR group (“breast cancer of all types”). In some embodiments, diagnoses are determined using the following criteria: the member has at least one inpatient claim associated with NEO030 CCSR diagnosis codes and/or the member has at least two distinct outpatient claims associated with NEO030 CCSR diagnosis codes. In other examples, a different CCSR code or combinations of CCSR codes are used to define a breast cancer diagnosis.

At step 206, the medical claim data associated with eligible members is split into training and validation datasets. As described above, the cohort datasets include CCSR (e.g., diagnostic) claim counts, pharmacy information, lab information, and biomarker data. Each eligible members is assigned a binary label indicating whether they had a breast cancer diagnosis in year n+1 or not. In some embodiments, the dataset of cohorts is split by 50-25-25% for training and two validation datasets respectively. In other examples, different dataset configurations are used (e.g., 40-30-30%, etc.). The distributions of healthy to breast cancer positive members is maintained when splitting up the datasets. In some embodiments, the training dataset (e.g., 50% of the input dataset) was down-sampled where the minority class (e.g, members with breast cancer) was left intact but the majority class (e.g., members without breast cancer) was under-sampled. In other words, the minority class was randomly selected until a training dataset having an equal (or substantially equal) number of minority class members and majority class members was achieved. In some examples, down-stream predictions with the validation datasets incorporated prediction probability adjustments to counter the under-sampling of the training dataset.

At step 208, feature selection is performed using the training dataset. In some embodiments, the feature (e.g., risk factor) selection process includes calculating one or more metrics associated with the training dataset. In some examples, the feature selection process includes determining the association of each risk factor type with a breast cancer diagnosis is calculated. For example, in some embodiments an odds analysis is performed to determine the probability of a breast cancer diagnosis for each CCSR type. In some embodiments, the odds (or probability) for each CCSR type is defined as the odds for having breast cancer at the end of an n-year prediction period (e.g., in year n+1) given a risk factor in the first year.

In some embodiments, the log odds for each CCSR type (or other risk factors) are calculated as follows: for each ccsrₙ in all n CCSRs, the number of claim counts associated with all diagnoses in the ccsrₙ is counted per eligible member. A unique count, m, is retained for each ccsrₙ. In certain examples, claim counts associated with fewer than a predetermined number of members (e.g., 5, 10, 15, etc.) were removed and considered outlier unique counts. For each unique count m of ccsrₙ, the probability of having a breast cancer diagnosis is calculated. This probability is equal to the number of members with a breast cancer diagnosis (member_{BC}) with unique count m of ccsrₙ divided by the total number of members. Several example claim counts and corresponding probabilities for a CCSR type are shown in Table 2 below:

TABLE 1

| Count | Probability |
|---|---|
| 0 | 0.00376356 |
| 1 | 0.00667946 |
| 2 | 0.00699239 |
| 3 | 0.00592869 |
| 4 | 0.00489425 |
| 5 | 0.00646552 |

The probabilities and claim counts in Table 2 correspond to the CCSR type END016 (“Other specified and unspecified nutritional and metabolic disorders”). For the CCSR type END016 there are 243 different diagnostic codes (e.g., ICD codes). The number of claims associated with any of these 243 diagnostic codes is counted per member. Then, for each unique count, the probability of having that count and having a breast cancer diagnosis in n years is calculated. In some embodiments, the natural log of the probabilities are taken to determine the log odds associated with each claim count per CCSR type.

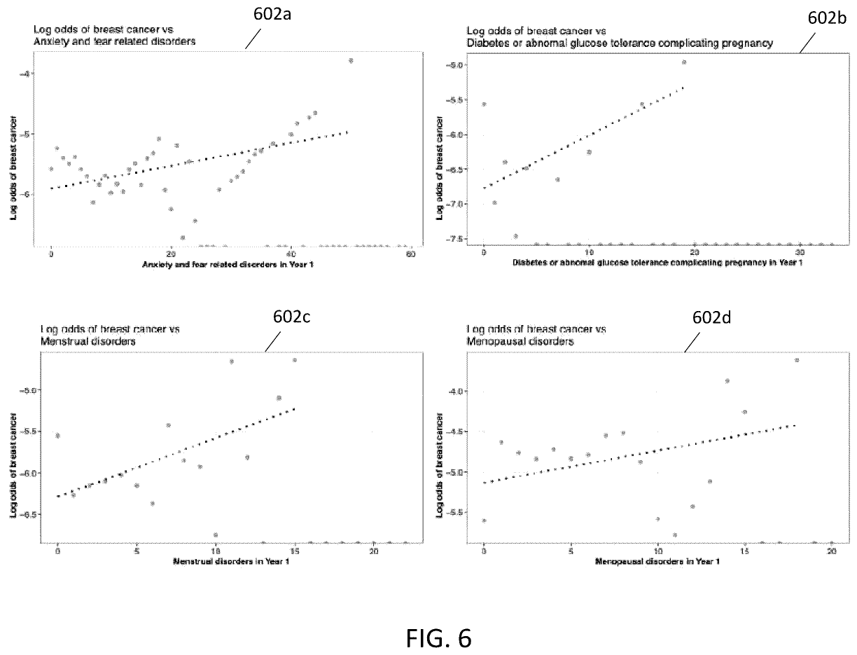

The log odds are used to determine trends (or associations) between each risk factor type and a breast cancer diagnosis. For example, FIG. 6 illustrates several example plots 602 of log odds for different CCSR types (e.g., characteristics, disorders, symptoms, diseases, etc.). In each plot, the x-axis represents the claim count and the y-axis represents the log odds of having a breast cancer diagnosis. In one example, the first plot 602a corresponds to the log odds of a breast cancer diagnosis relative to a first CCSR type (e.g., anxiety and fear related disorders), the second plot 602b corresponds to the log odds of a breast cancer diagnosis relative to a second CCSR type (e.g., diabetes or abnormal glucose tolerance complicating pregnancy), the third plot 602c corresponds to the log odds of a breast cancer diagnosis relative to a third CCSR type (e.g., menstrual disorders), and the fourth plot 602d corresponds to the log odds of a breast cancer diagnosis relative to a fourth CCSR type (e.g., menopausal disorders). As shown, each CCSR type has a different association (or correlation) to breast cancer diagnosis relative to claim count. In some examples, the risk factors that showed a positive trend with a breast cancer diagnosis were retained for model development.

In addition, the feature selection process includes calculating a variance inflation factor (VIF) and Akaike information criterion (AIC) with stepwise regression. In some embodiments, the VIF and AIC metrics are calculated for the risk factor type that passes the log odds analysis (e.g., showed a positive trend); however, in other examples, the VIF and AIC metrics are calculated for all risk factor types (e.g., all hypothesized risk factors).

In some examples, the VIF and AIC metrics are calculated to determine the multicollinearity of the training dataset. Multicollinearity defines a situation where input predictive features in a regression are linearly related to each other proving to be redundant. In some embodiments, redundancy increases a model's confidence intervals and reduce the effects of the independent variables. Additionally, independent individual predictors narrow the input space and allow for more reliable interpretation of input weights.

In some embodiments, VIF quantitatively identifies correlations between each predictor variable (e.g., risk factor). In some embodiments, high correlations are an indication that the input feature space is capable of being narrowed. The VIF metric uses a least squares regression analysis to determine collinearity across features. Several examples CCSR types and corresponding VIF values are shown in Table 3 below:

TABLE 3

| CCSR Type | VIF |
|---|---|
| END009 | 1.032840 |
| END016 | 1.007543 |
| FAC019 | 1.007561 |
| FAC021 | 1.024091 |
| GEN017 | 1.013314 |
| GEN021 | 1.018967 |
| GEN023 | 1.011029 |
| MBD005 | 1.021177 |
| MBD009 | 1.006985 |
| PRG008 | 1.513790 |
| PRG015 | 1.285816 |
| PRG019 | 1.107739 |
| PRG020 | 1.098173 |
| PRG028 | 1.310824 |
| PRG029 | 1.29901 |

In some embodiments, a VIF below a predetermined threshold (e.g., 3, 5, 7, etc.) indicates no significant collinearity for the corresponding risk factor type. In some examples, risk factor types with VIF values that exceed the predetermined threshold are eliminated from model development.

The second metric, AIC with stepwise regression, identifies the optimal combination of features while penalizing model complexity and input space size. The AIC stepwise regression provides an estimated coefficient, a standard error, a t-value, and a p-value for each risk factor type included. In some embodiments, the p-value of each risk factor is used to analyze the potential prediction contributions associated with each risk factor. Several example CCSR types and corresponding p-values determined from the AIC calculation are shown in Table 4 below:

TABLE 4

| CCSR Type | p-value |
|---|---|
| END016 | 0.002110 |
| FAC021 | <2e−16 |
| GEN017 | <2e−16 |
| GEN021 | <2e−16 |
| GEN023 | <2e−16 |
| MBD005 | 2.99e−11 |
| MBD009 | 0.032284 |
| PRG008 | 0.095707 |
| PRG015 | 0.027867 |
| PRG019 | 0.109423 |
| PRG028 | 0.000123 |
| PRG029 | <2e−16 |

In some embodiments, both forward and backward steps are included in the AIC calculation, where a regression model is built in a stepwise fashion, including and removing predictor variables (e.g., risk factor types) with each iteration. The output variables correspond to the “best” model. The predictor variables deemed significant by stepwise AIC and passed VIF were retained in the data. In some embodiments, risk factor types having a p-value below a predetermined threshold (e.g., 0.001, 0.01, etc.) are retained for model development. For example, in one embodiment based on the example data in Table 4, the CCSR types FAC021, GEN017, GEN021, GEN023, MBD005, PRG028, and PRG029 are retained for model development. In some embodiments, different metrics are used to determine which risk factor types are retained for model development (e.g., coefficients, standard errors, t-values, etc.).

At step 210, a machine learning (ML) model is trained using the training dataset and the selected predictor variables. In some embodiments, the ML model is a logistic regression model (or algorithm). In some embodiments, the ML model is one of a random forest model, a support vector machine(s) model, a decision tree(s) model, or a gradient boosted algorithm. In some embodiments, the logistic regression model is iteratively trained using a series of logistic regressions. For example, in some embodiments an initial logistic regression is performed using the training data. The initial logistic regression provides an estimated coefficient, a standard error, a z-value, and a p-value for each predictor variable. Several example predictor variables and corresponding p-values output from the initial logistic regression are shown in Table 5 below:

TABLE 5

| Predictor Variable | p-value |
|---|---|
| FAC021 | <2e−16 |
| GEN017 | <2e−16 |
| GEN021 | 1.54e−11 |
| GEN023 | <2e−16 |
| MBD005 | 0.03479 |
| PRG028 | 0.00938 |
| PRG029 | <2e−16 |

In some embodiments, predictor variables (e.g., risk factors) having a p-value below a predetermined threshold (e.g., 0.001, 0.01, etc.) are retained for model development. For example, in one embodiment based on the example data in Table 5, the CCSR types FAC021, GEN017, GEN021, GEN023, and PRG029 are retained for model development. In some embodiments, different metrics are used to determine which predictor variables are retained for model development (e.g., coefficients, standard errors, z-values, etc.).

In some embodiments, the log odds for each resulting predictor variable (e.g., each risk factor type that passed initial logistic regression) are recalculated with different transformations to determine best fit. In some embodiments, these transformations include square, log and/or natural log transformations. In some embodiments, a correlation coefficient (e.g., R-value) is used to determine the transformation with the best fit for each predictor variable.

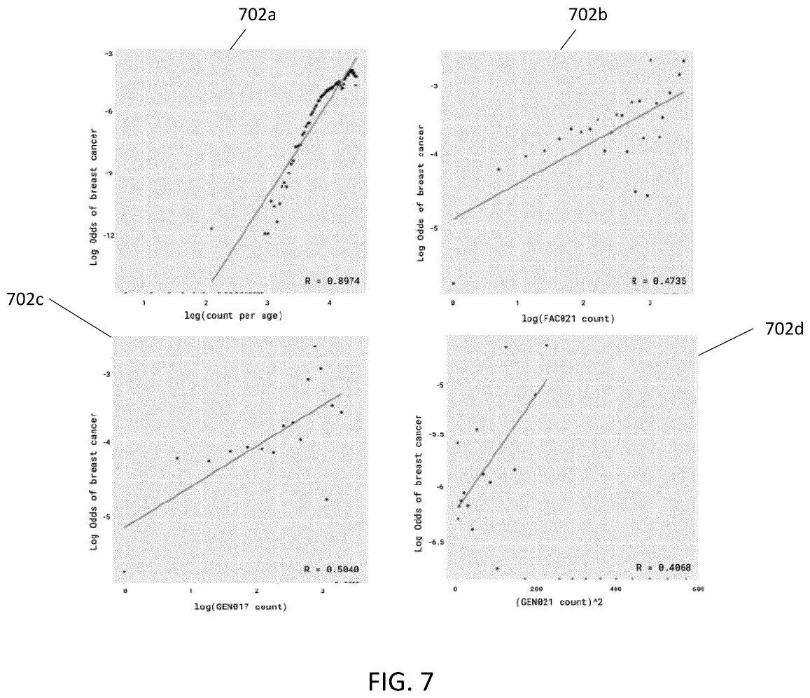

FIG. 7 illustrates several example plots 702 of log odds for different transformed predictor variables (e.g., characteristics, disorders, symptoms, diseases, etc.). In each plot, the x-axis represents the transformed claim count and the y-axis represents the log odds of having a breast cancer diagnosis. In one example, the first plot 702a corresponds to the log odds of a breast cancer diagnosis relative to a log transformation of the claim count of a first predictor variable (e.g., age), the second plot 702b corresponds to the log odds of a breast cancer diagnosis relative to a log transformation of the claim count of a second predictor variable (e.g., the FAC021 CCSR type), the third plot 702c corresponds to the log odds of a breast cancer diagnosis relative to a log transformation of the claim count of a third predictor variable (e.g., the GEN017 CCSR type), and the fourth plot 702d corresponds to the log odds of a breast cancer diagnosis relative to a square transformation of the claim count of a fourth predictor variable (e.g., the GEN021 CCSR type).

In some embodiments, a second logistic regression is performed using the training data and the transformed predictor variables. Similar to the initial logistic regression, the second logistic regression provides an estimated coefficient, a standard error, a z-value, and a p-value for each predictor variable. Several example predictor variables and corresponding p-values output from the second logistic regression are shown in Table 6 below:

TABLE 6

| Predictor Variable | p-value |
|---|---|
| FAC021 | <2e−16 |
| GEN017 | <2e−16 |
| GEN021 | 0.158 |

In some embodiments, predictor variables having a p-value below a predetermined threshold (e.g., 0.001, 0.01, etc.) are retained. For example, in one embodiment based on the example data in Table 6, the CCSR types FAC021 and GEN021 are retained as predictor variables. In some embodiments, different metrics are used to determine which predictor variables are retained for model development (e.g., coefficients, standard errors, z-values, etc.).

At step 212, the logistic regression model is tested with the first validation dataset. In some embodiments, the model is configured to output a breast cancer diagnosis prediction for each member included in the first validation dataset. In some embodiments, the resulting predicted probabilities are adjusted to account for the previous down-sampling of the training dataset. For example, in some embodiments the probabilities are adjusted using the following equations:

$$\\ \begin{matrix}scoring odds = \frac{predicted probabilities}{1 - predicted probabilities} & ( 1 )\end{matrix}$$

$$\\ \begin{matrix}adjusted odds = scoring odds \times \frac{original odds}{under - sampled odds} & ( 2 )\end{matrix}$$

$$\\ \begin{matrix}adjusted probability = \frac{1}{1 + ( \frac{1}{adjusted odds} )} & ( 3 )\end{matrix}$$

where, the predicted probabilities represents the output probabilities from the logistic regression model, the original odds represents the ratio of breast cancer members to healthy members included in the original dataset, and the under-sampled odds represents the ratio of breast cancer members to healthy members included in the training dataset. As described above, in some embodiments, the training dataset includes an equivalent proportion of classes (e.g., under-sampled odds=1).

At step 214, the adjusted prediction results corresponding to the first validation dataset are used to fine tune the logistic regression model. In some embodiments, the results are used to select cutoff and beta metrics for the resulting logistic regression model. The cutoff and beta metrics are referred to as “hyperparameters” of the logistic regression model. For example, in some embodiments selection is performed across a comparison of beta values 1 through 10 and cutoff thresholds between 0.01 and 1 at increments of 0.01. In some embodiments, selection is performed across a comparison of beta values of about 1 through about 10 and cutoff thresholds between about 0.01 and about 1 at increments of about 0.01. In some embodiments, different ranges and increments are used. The beta value and cutoff threshold that calculated the best f-measure was selected for the final version of the logistic regression model. Adjusted predicted probabilities greater than or equal to the cutoff were identified as increased breast cancer risk, and below as average or low breast cancer risk.

At step 216, the logistic regression model is updated. In some embodiments, the model is updated by adjusting (or optimizing) at least one hyperparameter of the model. For example, in some embodiments the logistic regression model is updated by applying the selected beta value(s) and/or the selected cutoff threshold(s) from step 214.

At step 218, the logistic regression model with the updated hyperparameters is tested with the second validation dataset. The model is configured to output a breast cancer diagnosis prediction for each member included in the second validation dataset. In some embodiments, the breast cancer diagnosis prediction is provided as a risk score. For example, in some embodiments the risk score is a grade, ranking, or percentage (e.g., B+, 90%, etc.). In some embodiments, a high score (e.g., A, 95%, etc.) indicates a low risk for breast cancer; however, in other embodiments, a high score indicates a high risk for breast cancer. The risk score is compared to one or more thresholds to determine a risk level (e.g., high risk, low risk, etc.) of each member.

In one example, the logistic regression model resulted in an accuracy of 89.53%, area under the curve (AUC) of 0.82, sensitivity of 0.39, and specificity of 0.90. In some embodiments, the logistic regression model resulted in an accuracy of approximately 89.53%, AUC of approximately 0.82, and sensitivity of approximately 0.39. In some embodiments, additional metrics such as observed-expected ratios and entropy metrics are used to assess model performance.

In some embodiments, once trained, the GRAM is used to provide breast cancer diagnosis predictions (e.g., risk scores) to users of the platform 100. For example, in some embodiments an input dataset corresponding to a user (e.g., patient, member) is provided to the platform 100 (or the risk assessment engine 106) to generate a user-specific breast cancer prediction. In some embodiments, based on the user-specific prediction (e.g., risk score), an alert or notification is automatically sent to the user, the user's physician, and/or the user's payor via the platform 100. It should be appreciated that in some embodiments, the GRAM is updated or retrained periodically by the risk assessment engine 106 as new data becomes available (e.g., data not included in the original training or validation datasets).

While described above as providing a bread cancer prediction score for users based on medical claim data, it should be appreciated that the risk assessment model (e.g., the GRAM) may be advantageously used for various other applications. For example, in some embodiments the GRAM may be used to scan for other types of cancers or diseases. In some embodiments, the algorithms, concepts, and/or techniques used to build, develop, and train the risk assessment model for breast cancer are applied to other cancers such as endometrial or ovarian cancers, or applied to preventable or difficult to diagnose syndromes such as polycystic ovary syndrome.

In some embodiments, the risk assessment calculator (e.g., the GRAM) is offered to users outside of specific payor partnerships by setting up a fee for access to the calculator and subsequent community/resource access. In some embodiments, the GRAM becomes part of the standard of care for all women at the first sign of any breast pathology or part of the regular pap smear exam schedule. The GRAM could help women without the ultimate end user needing to know about and seek the GRAM platform. Full integration of the GRAM calculator would allow for the least amount of disruption and most impact in women's lives. In some embodiments, the GRAM could also be integrated into hospital/clinic EMRs such that physicians and/or nurse practitioners can order or prescribe the GRAM calculator be run for a particular patient.

In some embodiments, the GRAM (or platform 100) is leveraged to gain an understanding of how many women are scheduled for but not booking/attending their prescribed breast cancer screening examinations (e.g. mammogram, MRI, ultrasound, etc.). This information could not only help providers ensure that their patients are getting screened when they need to be but will also help payor to increase revenue.

### Topic Modeling

In some embodiments, to calculate a better understanding of the claims data and derive “signatures” associated with specific classes, the platform 100 (or another computing device or system) is configured to use a Latent Dirichlet Allocation (LDA) based model to provide a probabilistic topic modeling approach. The LDA model assumes latent features are associated with different classes. For example, in some embodiments the LDA model assumes that features—clinical data, RX information, personal family history, etc.—are driven by the different classes. In this case, the classes being breast cancer positive and breast cancer negative (e.g., healthy), as well as different stages of breast cancer across each other.

In the context of topic modeling, LDA models are given a set of documents about several topics. From those documents the model derives a set of topics where each topic is associated with multiple words. Those topics are correlated with each document based on proportions. In some embodiments, the platform 100, when deriving “signatures” associated with specific classes uses inputs and outputs. The inputs include “documents with words.” For example, a digital media company uses topic modeling to understand what content their readers/users like to take in by assessing a customer's past content (e.g., input) and then using the topics generated (e.g., output) to suggest relevant stories/content for future consumption.

In a use case of the platform 100, members with breast cancer are treated as a first document and members without breast cancer are treated as a second document. The associated claims data and social determinants of health are the “words” of each document. In another use case of the platform 100, members with different stages of breast cancer are treated as different documents. The associated claims data and social determinants of health are the “words” of each document. The resulting topic associations are combined using ensemble learning, resulting in a predictive claims data “signature”.

In some embodiments, the “words” are the diagnoses (e.g., ICD, CCSR codes), procedures (e.g., CPT codes), demographics, lab information, pharmacy information, and other information provided by the user and payor. The “documents” with those “words” are fed into the LDA model or algorithm. The output is a series of topics with “words”. A quantitative association between topics and documents (e.g., topics being correlated member information and documents being breast cancer stages) is calculated.

Table 7 below is an example of “topics” and potential “words” associated with such “topics” to illustrative this concept further:

TABLE 7

| Topic | “Words” | Type |
|---|---|---|
| Topic 1 | ICD codes related to obesity | Clinical |
| | ICD codes related to | |
| | metabolic disorders | |
| Topic 2 | ICD codes related to anxiety | Clinical |
| | and depression | |
| | RX information related to | |
| | mood disorders | |
| Topic 3 | Line of business correlated | Socioeconomic |
| | with lower middle class | |
| Topic 4 | Line of business related to | Socioeconomic |
| | upper middle class | |
| | Products with more features | |
| | and associated with larger | |
| | employers | |
| Topic 5 | Demographic A, B, C, D | Demographic |
| Topic 6 | Demographic E, F, G, H | Demographic |
| Topic 7 | Demographic A, ages 10-15, | Demographic |
| | 16-20, 21-25 | |
| Topic 8 | Demographic B, C, D ages | Demographic |
| | 20-25, 26-30 | |

These topics and associated weights constitute the “breast cancer signature” and the “null signature”. In some embodiments, the LDA workflow is performed without separating the types of features—input all features without a label of personal, familial, clinical, socioeconomic, or demographic.

### Alternative Risk Assessment Techniques

As described above, the risk assessment model (e.g., the GRAM) is a logistic regression machine learning model configured to provide risk assessment scores. However, in some embodiments, different types models and/or algorithms are used to build and develop the risk assessment model. For example, in some embodiments the model uses a deep learning backbone (e.g., a neural network). The model may correspond to a deep learning workflow that leverages nonlinear machine learning modalities to identify the most influential features and integrate them in a weighted fashion. This workflow uniquely adapts multi-omic and computer vision techniques in a clinical setting (e.g., neural networks, ensemble machine learning, and image masking and saliency). The deep learning model is built to be adaptable (e.g., consistently finetuned and improved with each new dataset).

In some embodiments, the platform 100 includes a neural network which is constructed with each layer corresponding to sets of related risk factors. One or more factors are selected based on overlapping features from a subset of the Sandbox, UK Biobank, nurses health study, and other publicly available datasets. Cross validation is incorporated with each iteration to track prediction errors and prevent overfitting. Image masking—a sensitivity analysis used to quantitatively describe the impact that each input component has on the end prediction—is adapted for each feature, called feature masking. In some embodiments, individual and combinations of up to three features per individual are retained and used as input in the fully trained expanded model.

The final adjusted risk assessment is compared to the original assessment for that individual using divergence metrics. Features are ranked based on divergence metrics to determine rank of influence. In some embodiments, the biological implication of these features including novel application of combined clinical, social determinant, demographic, and/or lifestyle-based factors are validated by clinical and research experts and published literature.

In some embodiments, the final adjusted risk assessment is compared to or combined with existing models (e.g., Gail model, TC model, etc.). For example, in some embodiments through the use of ensemble machine learning techniques, the expanded model and combined model with the Gail and TC models is incorporated using the remaining subset of the participant data (e.g., Sandbox, UK Biobank, nurses' health study, and other publicly available sets). In some embodiments, shifts in the overall risk assessment between the weighted Gail and TC models, the expanded model, and the combined model is assessed through AUC changes. In some embodiments, noise and entropy on final predictions across models is quantified by divergence metrics and the final GRAM is based on the model with the highest accuracy and least entropy.

In some embodiments, the risk assessment model includes an autoencoder that is used to learn a distribution of features in the claims data and general associations with classes (e.g., breast cancer or healthy). In some embodiments, the autoencoder is used by the platform 100 when determining a risk assessment. The autoencoder takes in or receive a user's claims data (e.g., a user may refer to a member of an insurance policy) as input, compress the information, and then decompress the information to reconstruct the data. Through training, the autoencoder determines weights that are used to efficiently compress and reconstruct the claims data. The trained autoencoder weights are used to expedite the training of downstream models (e.g., the claims model) by providing a baseline for the parameters instead of starting with random parameters. This allows the claims model to begin training with a set of learned parameters. In some embodiments, the trained autoencoder weights are also used as non-random weight initialization and/or frozen weights in the claims model. In some embodiments, the weights correspond to parameters that weigh features (e.g., input features).

In some embodiments, the autoencoder narrows the feature space and expedite the training of the claims model by providing a baseline for parameter values instead of starting at random (e.g., with a large number of parameters and/or values). The claims model is then trained with claims data (e.g., from insurance companies/payors) and the weights (e.g., parameters) derived from the autoencoder. In some embodiments, debiasing and generalizability are used to train the claims model to make the claims model more generalizable. The output of the claims model is a risk assessment model that is used to predict breast cancer diagnosis in users (e.g., provide a breast cancer risk score).

The output from the claims model is interpreted using two methods—masking and saliency. In some embodiments, both of these methods occur simultaneously and provide quantitative insight on the association of each feature with a breast cancer diagnosis. These interpretation methods allow for biological/literature validation and downstream clinical validation. In some embodiments, an alternative to the claims model and interpretation steps includes using topic modeling (e.g., alternative information). This method derives signatures that are used as the predictive model (e.g., instead of the neural networks and machine learning in the claims model).

In some embodiments, the claims model is configured to determine associations between the features from the claims data and breast cancer diagnoses. In some embodiments, breast cancer positive is determined by either the presence of two breast cancer diagnosis claims and/or the presence of a pathology related breast cancer code. If a member is breast cancer positive based on this criterion, then the first instance of a breast cancer suspicion on their claims record was used as the platform 100's target. In some embodiments, this suspicion includes any procedure code used for breast cancer diagnosis and screening immediately before a breast cancer diagnosis code, presence of diagnosis code pertaining to a benign breast nodule, breast lump, and/or related breast anomaly.

In some embodiments, the claims model is configured as a “risk factor type model.” As such, the claims model analyzes the medical claims dataset as subsets of groupings based on groups of risk factors. In some embodiments, these groups include: personal factors (e.g., age, height, weight, place of birth, race, gender identity), socioeconomic factors (e.g., income, education, employment, zipcode, marital status, etc), and/or clinical and medical factors (e.g., known genetic risks, underlying conditions, etc.).

Individual models are developed from risk factor groups. For example, in some embodiments personal factors (e.g., familial history, lifestyle, age, height, weight, etc), is one subset of the data with all patients included. This is fed into a feed forward neural network to result with a classification model to identify probability of a breast cancer diagnosis by stage. To assist in training, factor weights from the optimized autoencoder are transferred—resulting in models that have the same encoding architecture, activation function, and optimizer. Additionally, instead of a random initializer, the starting factors are based on the encoding architecture.

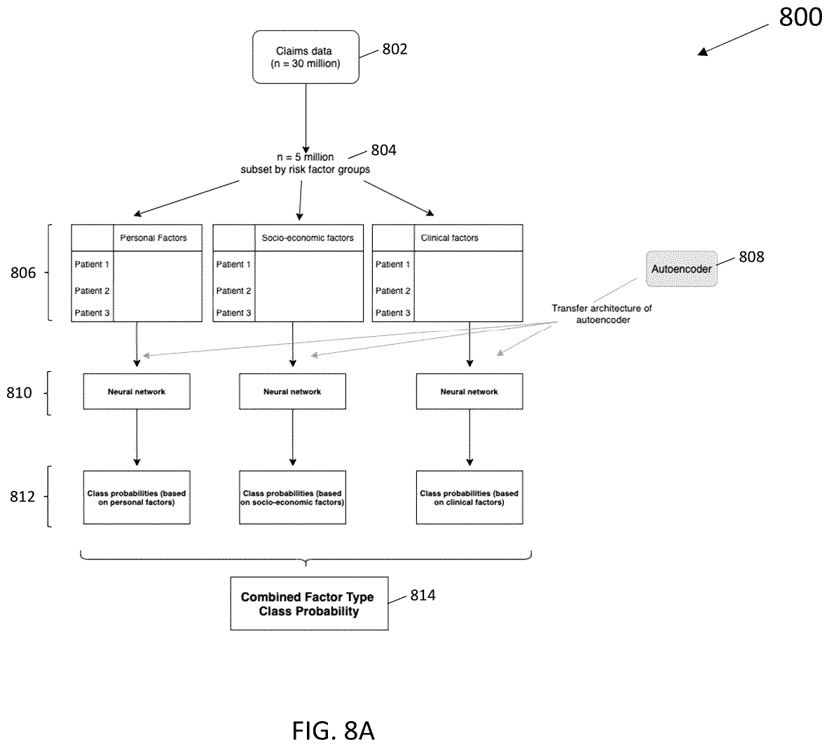

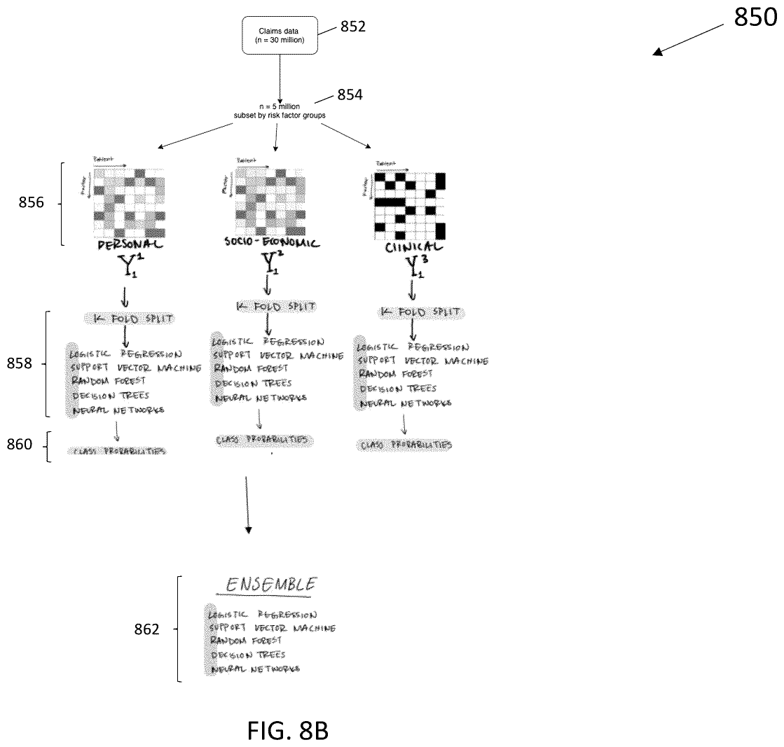

FIG. 8A illustrates a method 800 for developing and training a claims model in accordance with aspects described herein. In some embodiments, the method 800 corresponds to the development and training of a risk factor type model.

At step 802, medical claims data is received and preprocessed where columns are all factors and rows are all members (or patients). In some embodiments, the medical claims data includes at least 30 million claims.

At step 804, the distribution of each of the factor types (e.g., personal, socio-economic, clinical, etc.) is taken and a subset is extracted—maintaining the same distribution. In some embodiments, final subset dataset includes at least 5 million claims per factor type.

At step 806, the subset data is segregated by factor type with columns as features and rows and members.

At step 808, the encoding architecture from the autoencoder is incorporated with the presence of a breast cancer diagnosis or not (e.g., healthy) as the target.

At step 810, each factor type is fed into a corresponding feed-forward neural network. In some embodiments, each factor type is fed into a common neural network.

At step 812, the neural networks provide a class probability for each member.

At step 814, the class probabilities for each factor are combined to provide a risk assessment score for each member.

FIG. 8B illustrates another method 850 for developing and training a claims model in accordance with aspects described herein. In some embodiments, the method 850 corresponds to the development and training of a risk factor type model.

At step 852, medical claims data is received and preprocessed where columns are all factors and rows are all members (or patients). In some embodiments, the medical claims data includes at least 30 million claims.

At step 854, the distribution of each of the factor types (e.g., personal, socio-economic, clinical, etc.) is taken and a subset is extracted—maintaining the same distribution. In some embodiments, final subset dataset includes at least 5 million claims per factor type.

At step 856, the subset data is segregated by factor type with columns as features and rows and members.

At step 858, he data is fed into different machine learning models and/or algorithms. For example, in some embodiments the data for each factor type is provided to a k-fold split model (e.g., ¾ training and ¼ testing). The training data from the k-fold split model is used with a logistic regression model, a support vector machine model, a random forest model, a decision tree model, or any other suitable machine learning model or algorithm. In some embodiments, the best performing algorithm is selected per factor type dataset. For example, in some embodiments the best performing algorithm is selected using metrics such as accuracy, AUC, sensitivity, and/or specificity.

At step 860, the class probability for breast cancer risk prediction based on factor type is provided.

At step 862, the resulting models (e.g., one model per factor type) are combined using stacked generalization or ensemble learning—involving combining multiple weak classifiers to boost prediction accuracy, sensitivity, and specificity. In some embodiments, a weighted combination of the individual factor type models where the weights are the coefficients of the ensemble model is provided.

In some embodiments, the claims model is configured as an “individual risk factor model.” As such, the claims model analyzes the medical claims dataset as subsets of groupings based on masking one individual risk factor. For example, in some embodiments if there are n factors in the claims dataset, the first subset consists of all factors-factor₁ and further delineated with all members grouped by categorical features of factor₁. The second subset consists of all factors-factor₂ with all members grouped by categorical features of factor₂ and so on with subsets consisting of all factors-factorₙ and members grouped by categorical features of factorₙ. In some embodiments, one factor is excluded at a time and then followed by grouping the members by that excluded factor to account for biases prior to combining (or ensembling) the models.

For example, in some embodiments if age was removed as a factor, the result is a data set of all factors-factor_{age}. From here, the dataset is subset further based on member age. Each of the subsets have the same columns (factors), but the members for each subset is different. A first subset has members ages up to 12 (or about 12), a second subset has members ages 13-18 (or about 13-18), a third subset has members ages 19-24 (or about 19-24), and so on. In some embodiments, a new member is run through all the models of all factors-factor_{age} for each age bin. The differences in risk assessment across models indicates quantitatively how much of an effect age plays in the model development as well. This indicates whether individual models should be built with age categorized and separated then ensembled or not. This same process is repeated for all factors and different combinations of factors as well.

The individual claims model is developed from a subset based on an individual's risk. For example, in one embodiment all patients of ages 15-35, 36-55, 56-75, 76 and above, are all individual subsets resulting in individual models. In some embodiments, each model is fed into feed forward neural networks to provide a classification model that identifies the probability of a breast cancer diagnosis by stage. To assist in training, factor weights from the autoencoder are transferred—resulting in models that have the same encoding architecture, activation function, and optimizer. Additionally, instead of a random initializer, the starting factors are based on the encoding architecture.

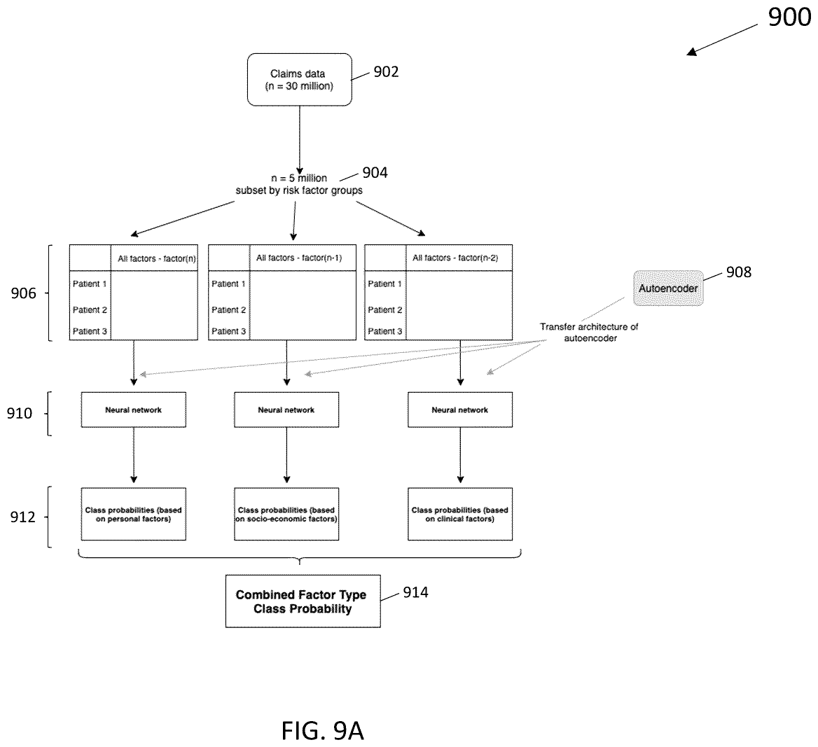

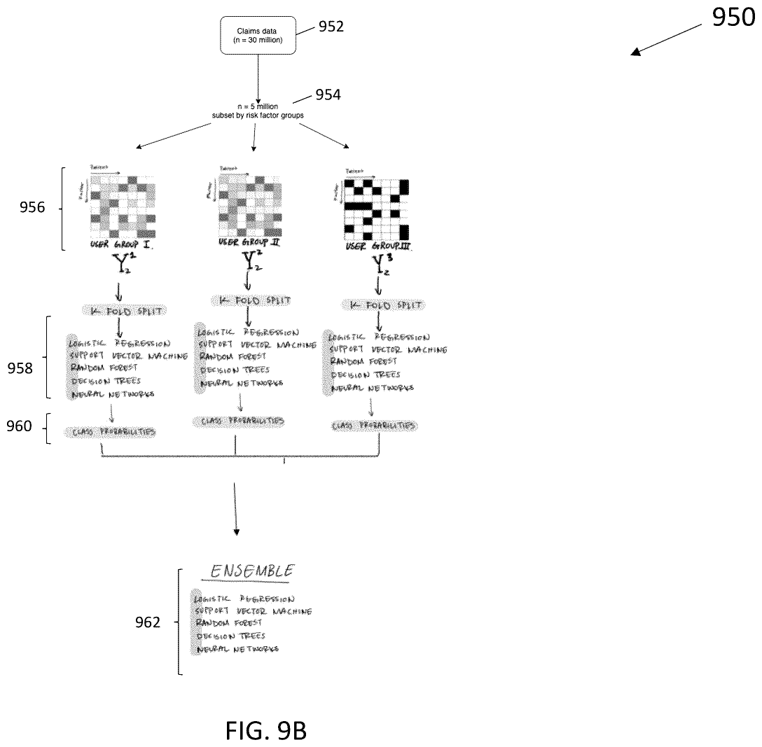

FIG. 9A illustrates a method 900 for developing and training a claims model in accordance with aspects described herein. In some embodiments, the method 900 corresponds to the development and training of an individual risk factor model.

At step 902, medical claims data is received and preprocessed where columns are all factors and rows are all members (or patients). In some embodiments, the medical claims data includes at least 30 million claims. In some embodiments, the medical claims data includes about 30 million claims.

At step 904, the distribution of each of the factor types (e.g., personal, socio-economic, clinical, etc.) is taken and a subset is extracted—maintaining the same distribution. In some embodiments, final subset dataset includes at least 5 million claims per factor type. In some embodiments, the final subset dataset includes about 5 million claims per factor type.

At step 906, the dataset is split into n datasets where n represents the total number of factors. Each dataset contains all but one of the factors. The removed factor is used to categorically group the members. Therefore, the columns of the first dataset consists of all factors-factor₁ with members grouped by factor₁, the second consists of all factors-factor₂ with members grouped by all factors-factor₂, and so on.

At step 908, the encoding architecture from the autoencoder is incorporated with the presence of a breast cancer diagnosis or not (e.g., healthy) as the target.

At step 910, each factor group is fed into a corresponding feed-forward neural network. In some embodiments, each factor group is fed into a common neural network.

At step 912, the neural networks provide a class probability for each member.

At step 914, the class probabilities for each factor group are combined to provide a risk assessment score for each member.

FIG. 9B illustrates another method 950 for developing and training a claims model in accordance with aspects described herein. In some embodiments, the method 950 corresponds to the development and training of an individual risk factor model.

At step 952, medical claims data is received and preprocessed where columns are all factors and rows are all members (or patients). In some embodiments, the medical claims data includes at least 30 million claims. In some embodiments, the medical claims data includes about 30 million claims.

At step 954, the distribution of each of the factor types (e.g., personal, socio-economic, clinical, etc.) is taken and a subset is extracted—maintaining the same distribution. In some embodiments, final subset dataset includes at least 5 million claims per factor type. In some embodiments, the final subset dataset includes about 5 million claims per factor type.

At step 956, the dataset is split into n datasets where n represents the total number of factors. Each dataset contains all but one of the factors. The removed factor is used to categorically group the members. Therefore, the columns of the first dataset consists of all factors-factor₁ with members grouped by factor₁, the second consists of all factors-factor₂ with members grouped by all factors-factor₂, and so on.

At step 958, the data is fed into different machine learning models and/or algorithms. For example, the data for each factor type is provided to a k-fold split model (e.g., ¾ training and ¼ testing). In some embodiments, the training data from the k-fold split model is used with a logistic regression model, a support vector machine model, a random forest model, a decision tree model, or any other suitable machine learning model or algorithm. In some embodiments, the best performing algorithm is selected per factor dataset. For example, in some embodiments the best performing algorithm is selected using metrics such as accuracy, AUC, sensitivity, and/or specificity

At step 960, the class probability for breast cancer risk prediction based on factor group is provided.

At step 962, the resulting models (e.g., one model per factor group) are combined using stacked generalization or ensemble learning—involving combining multiple weak classifiers to boost prediction accuracy, sensitivity, and specificity. In some embodiments, a weighted combination of the individual factor group models where the weights are the coefficients of the ensemble model is provided.

In some embodiments, the platform 100 utilizes interpretation to determine a risk assessment model. For example, in some embodiments, the platform 100 utilizes “factor masking.”

Image masking is generally a type of computer vision analysis where a mask is applied to a portion of an image (e.g., setting all pixels in that region to 0) while retaining the rest of the image. This is a sensitivity analysis to quantitatively determine the impact of the retained image on the final classification. In this case, the platform 100 uses what is termed as “factor masking.” The platform 100 performs two types of factor masking: masking groups of risk factors (e.g., factor types), and masking of the individual risk factors. The purpose of factor type masking is to quantitatively determine the influence of a group of risk factors (e.g. clinical, personal, socioeconomic, etc.) to breast cancer risks. Similarly, the purpose of individual risk factor masking is to quantitatively determine the influence of association of individual factors (e.g., age, height, place of birth, risk gene mutations, types of underlying conditions, etc.) on breast cancer risks. In some embodiments, masking underlies and defines the associations developed from the claims model—solving the problem of black box interpretability of neural networks and deep learning.

In some embodiments, risk factor masking is performed by grouping the input dataset based on factor types wherein fₙ is the individual factor and F_{M} indicates the factor type it belongs to (e.g. personal factors F_{P}, clinical factors F_{C}, socioeconomic factors F_{S}, etc.). The data is organized such that the factor types are the columns and patients are the rows. One of the factor types is retained while the rest are masked (e.g., set to zero). The masked data is fed as the input into the trained claims model resulting in predictions for each patient based on solely the retained factor type. This process is repeated for each factor type F_{M}. The amount of change between the predicted risk assessment from the full dataset for a patient versus the predicted risk assessment from the masked dataset for a patient is determined using average precision (AP) and AUC metrics and is calculated per F_{M}. Using AP and AUC metrics, the factor types are ranked to determine the strength of influence of the risk assessment predictions. The ranked list of factor types indicates defined associations with breast cancer risk. The list exposes factor types that have yet to be explored in the context of cancer risk from a quantitative perspective.

In some embodiments, a similar process is performed for individual risk factor masking. For example, in some embodiments the input dataset is grouped based on factor types wherein fₙ is the individual factor. The data is organized such that the factors are the columns and patients are the rows. One of the factors is retained while the rest are masked (e.g., set to zero). In some embodiments, all factors are set to 0 except for one. The masked data is fed as the input into the trained claims model resulting in predictions for each patient based on solely the retained factor type. This process is repeated for each factor fₙ. The amount of change between the predicted risk assessment from the full dataset for a patient versus the predicted risk assessment from the masked dataset for a patient is determined using AP and AUC metrics and is calculated per fₙ. Using the AP and AUC metrics, the factors are ranked to determine the strength of influence of the risk assessment predictions. The ranked list of factors defines quantitative associations with breast cancer risk. The list exposes factors that have yet to be explored as well in the context of cancer risk from a quantitative perspective.

In some embodiments, both of these masking analyses are used to validate the association results from the claims model. Additionally, insights from the calculated interpretations dictate where and what unbiasing steps are required.

The platform 100 also utilizes class saliency to better determine a risk assessment model. Class saliency is another computer vision technique that is used to compute the gradient of an output class prediction with respect to an input via back propagation. Thus, class saliency is used by the platform 100 to identify the relevant input components whose values would affect the positive class probability in a trained neural network. In some embodiments, the platform 100 uses what is termed as “factor saliency” to be the factors whose change in value would increase the model's belief of the positive class label. In this case, the more salient factors were the ones that increased the probability of developing a disorder or disease, such as the probability of developing breast cancer.

### Unbiasing (De-Biasing)

In some embodiments, machine learning models and algorithms, when implemented, generate bias. In some embodiments, the machine learning features of the platform 100 generate bias over time. In an effort to de-bias (or unbias) the platform 100, a de-biasing technique is employed by the platform 100.

In some embodiments, a de-biasing technique is implemented using generative adversarial networks (GANs). Generative Adversarial Networks (GANs) are unsupervised learning tasks in machine learning that involve two sub-models: a generator model, which creates new plausible data examples from the original data and a discriminator model, which classifies the data (both new and original) as real (e.g., original) or fake (e.g., new). These GANs learn the “ins and outs” of the dataset and generate new data points that would plausibly be a part of the original set. The modified datasets are ran as a supervised learning problem and the discriminator model attempts to identify examples as either “real” (e.g., from the original data) or “fake” (e.g., from the generated set). The two sub-models are trained together until the model is not able to tell the two groups apart (e.g., 50% of the time or about 50% of the time). This means that the model is generating truly plausible examples.

The use of GANs enable the platform 100 to decrease the bias introduced to the platform 100's risk assessment model (e.g., the GRAM model) by the datasets. In some embodiments, many marginalized populations within the platform 100's datasets are not as well represented as others (i.e., Caucasian, middle-class, and over age 45). By implementing GANs, the platform 100 evens out the representation and create a risk assessment model that consciously tackles bias from all angles, ensuring that the platform 100 is capable of providing all women (or men) with an accurate risk assessment regardless of age, ethnicity, socio-economic status, and all other differentiators.

### Dynamic Sampling

In some embodiments, dynamic sampling of training data and ranked latent bias variables using debiased variational autoencoders is employed by the platform 100. In such examples, the method of de-biasing uses the learned representations of the model to adjust the training data. Adjustments are based on where the model fails or significant variance accuracy results for certain demographics or groups of members that have a lack of dimensionality in the training data. This is an unsupervised approach where the platform 100 does not specify the underrepresented groups (e.g., sensitive features). The unbalanced representations are learned during the training process. For example, in some embodiments when deciding if a member has breast cancer or not, latent variables (e.g., such as demographics or geographical features) are not drastically impact the classification results. The idea is that in a class (e.g., breast cancer verses healthy), all latent variables need to be balanced. Balancing these variables requires learning the structure of the latent variables.

Variational autoencoders (VAE) are also be used by the platform 100 to learn the latent variable structures. This is achieved in a semi-supervised fashion. The workflow involves the VAE learning the true distribution of the latent variables. During this process, the platform 100 would indicate the supervised output variables (hence semi-supervised with the unsupervised latent variables). In this case, the VAE learns the latent variable structure for one class at a time (e.g., breast cancer members and healthy population). Next, an overall workflow to create the debiased training dataset is decided by: 1) latent distribution estimated through the encoder and 2) adaptively adjust the frequency of the occurrence of over represented regions and increasing sampling of underrepresented regions. In some embodiments, the training dataset is used by the platform 100 to train the risk assessment (or claims) model.

In some embodiments, this method provides insight on which latent variables provide the most bias in the original algorithm based on the amount of adjustment used per variable. The adjustment amount is used to rank the latent variables based on the variables introducing the most bias. This ranked list of latent variables used to develop individual risk models per member population associated with each variable. For example, in some embodiments if demographic variables are highly ranked to bias the algorithm altogether, then the platform 100 develops individual demographic groups of members using the same claims model approach. This results in multiple models per demographic group which is then combined using ensemble learning (e.g., similar to the ensemble method in claims model). The purpose of ensembling the individual bias models is to accommodate for new members who fall under multiple categories.

### Generalizability

In some embodiments, transfer learning using autoencoders for generalizability is employed by the platform 100. Transfer learning is a method where information gained from training one model is transferred to a different problem space. This decreases the time needed for training each subsequent problem and sample size. The platform 100 utilizes pre-trained layers of machine learning networks and add subsequent dense layers at the end for recognizing members of the new dataset. In some embodiments, the risk assessment model is initially created, trained, and tested with data from a first payor. Subsequently, using transfer learning techniques, the model is then be adapted for a second payor, a third payor space, etc. The goal is to create additional layers in the machine learning approach associated with each individual payor and the unique information they provide while also tuning the existing overlapping layers.

In some embodiments, generalizability using ensembling of multiple models using overlapping, dataset specific, and end user features is employed by the platform 100. For example, a first level (e.g., Level 1) corresponds to features overlapping with most payors, a second level (e.g., Level 2) corresponds to payor specific features, and a third level (e.g., Level 3) corresponds to user survey features.

In some embodiments, the risk assessment model (e.g., the GRAM) is trained and tested using data from a first payor to develop three model levels. The first model (e.g., Level 1) contains features that are overlapping across most national payors—diagnoses, procedures, labs, pharmacy, and basic member information (e.g., age, height, weight). The second model (e.g., Level 2) contains features specific to the payor (e.g., the payor associated with the input dataset). Payor specific information is generally varying in social determinants of health information. The third model (e.g., Level 3) contains features that are from end user surveys. For example, in some embodiments the end user surveys include information such as: “at what age did the member start their first period?”, “did the member breastfeed after giving birth and for how long?”, “at what age did they start menopause?”, and other information that is not available from claims data.

The three levels are created and trained with a first payor's claims data and employees under the first payor (e.g., who are the end users). The levels are ensembled to create a final risk assessment prediction model. Next, the model is taken to a second payor and fined tuned with second payor's data and end users. As such, there are four models: Level 1_{retuned}, Level 2_{first_payor}, Level 2_{second_payor}, and Level3_{retuned}. In some embodiments, all four models are ensembled to provide a combined model. This approach creates a “generalizable” model. This model is then be used across users with any insurance, and without insurance, since it has levels pertaining to multiple types of information.

As described above, systems for methods for providing a comprehensive data analytics platform that virtually assesses a user's risk of developing a disease (e.g., cancer) is described herein. In at least one embodiment, clinical risk assessments and genetic risk assessments are combined to improve risk analysis of a particular disease (e.g., cancer), medical condition, and/or syndrome. In some embodiments, machine learning models and algorithms are utilized to determine such a risk assessment of a user (e.g., human female or human male).

### Hardware and Software Implementations

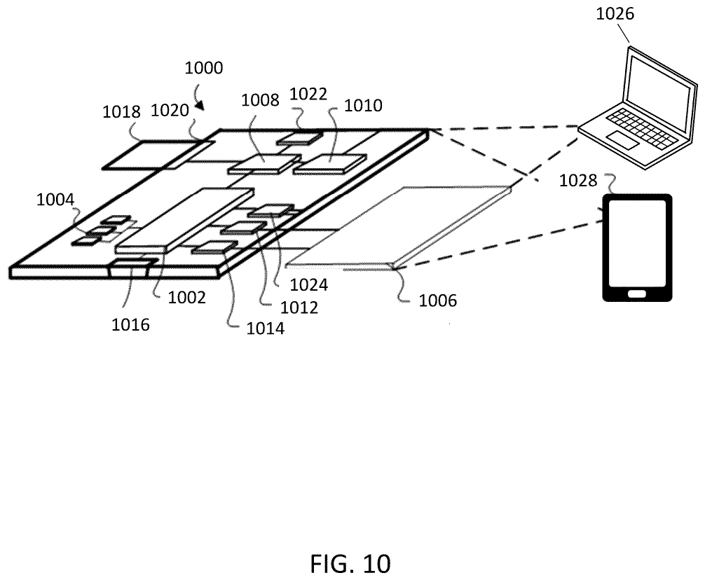

FIG. 10 shows an example of a generic computing device 1000, which may be used with some of the techniques described in this disclosure. Computing device 1000 includes a processor 1002, memory 1004, an input/output device such as a display 1006, a communication interface 1008, and a transceiver 1010, among other components. The device 1000 may also be provided with a storage device, such as a micro-drive or other device, to provide additional storage. Each of the components 1000, 1002, 1004, 1006, 1008, and 1010, are interconnected using various buses, and several of the components may be mounted on a common motherboard or in other manners as appropriate.

The processor 1002 can execute instructions within the computing device 1000, including instructions stored in the memory 1004. The processor 1002 may be implemented as a chipset of chips that include separate and multiple analog and digital processors. The processor 1002 may provide, for example, for coordination of the other components of the device 1000, such as control of user interfaces, applications run by device 1000, and wireless communication by device 1000.

Processor 1002 may communicate with a user through control interface 1012 and display interface 1014 coupled to a display 1006. The display 1006 may be, for example, a TFT LCD (Thin-Film-Transistor Liquid Crystal Display) or an OLED (Organic Light Emitting Diode) display, or other appropriate display technology. The display interface 1014 may comprise appropriate circuitry for driving the display 1006 to present graphical and other information to a user. The control interface 1012 may receive commands from a user and convert them for submission to the processor 1002. In addition, an external interface 1016 may be provided in communication with processor 1002, so as to enable near area communication of device 1000 with other devices. External interface 1016 may provide, for example, for wired communication in some implementations, or for wireless communication in other implementations, and multiple interfaces may also be used.

The memory 1004 stores information within the computing device 1000. The memory 1004 can be implemented as one or more of a computer-readable medium or media, a volatile memory unit or units, or a non-volatile memory unit or units. Expansion memory 1018 may also be provided and connected to device 1000 through expansion interface 1020, which may include, for example, a SIMM (Single In Line Memory Module) card interface. Such expansion memory 1018 may provide extra storage space for device 1000, or may also store applications or other information for device 1000. Specifically, expansion memory 1018 may include instructions to carry out or supplement the processes described above, and may include secure information also. Thus, for example, expansion memory 1018 may be provided as a security module for device 1000, and may be programmed with instructions that permit secure use of device 1000. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1004, expansion memory 1018, memory on processor 1002, or a propagated signal that may be received, for example, over transceiver 1010 or external interface 1016.

Device 1000 may communicate wirelessly through communication interface 1008, which may include digital signal processing circuitry where necessary. Communication interface 1008 may in some cases be a cellular modem. Communication interface 1008 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1010. In addition, short-range communication may occur, such as using a Bluetooth, WiFi, or other such transceiver (not shown). In addition, GPS (Global Positioning System) receiver module 1022 may provide additional navigation- and location-related wireless data to device 1000, which may be used as appropriate by applications running on device 1000.

Device 1000 may also communicate audibly using audio codec 1024, which may receive spoken information from a user and convert it to usable digital information. Audio codec 1024 may likewise generate audible sound for a user, such as through a speaker, e.g., in a handset of device 1000. Such sound may include sound from voice telephone calls, may include recorded sound (e.g., voice messages, music files, etc.) and may also include sound generated by applications operating on device 1000. In some examples, the device 1000 includes a microphone to collect audio (e.g., speech) from a user. Likewise, the device 1000 may include an input to receive a connection from an external microphone.

The computing device 1000 may be implemented in a number of different forms, as shown in FIG. 10. For example, it may be implemented as a computer (e.g., laptop) 1026. It may also be implemented as part of a smartphone 1028, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

### Terminology

The phrasing and terminology used herein is for the purpose of description and should not be regarded as limiting.

Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, one skilled in the art shall recognize that: (1) certain steps may optionally be performed; (2) steps may not be limited to the specific order set forth herein; (3) certain steps may be performed in different orders; and (4) certain steps may be performed simultaneously or concurrently.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The term “about” as used in the specification and the claims (e.g., “X has a value of about Y” or “X is about equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of” or “exactly one of” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A computer-implemented method for training a machine learning (ML) model to assess the risk of a human subject for developing at least one disorder, the method comprising:
receiving an input dataset including at least medical claim data corresponding to a plurality of human subjects over a target prediction period;
splitting the input dataset into a first dataset corresponding to a first portion of the plurality of human subjects and a second dataset corresponding to a second portion of the plurality of human subjects;
selecting at least one risk factor associated with developing the at least one disorder from the first dataset;
training a machine learning (ML) model using the first dataset and the at least one risk factor, the ML model including at least one logistic regression model;
providing the second dataset to the ML model to generate a risk prediction for developing the at least one disorder by the end of the target prediction period for each human subject included in the second portion of the plurality of human subjects; and
tuning at least one parameter of the ML model based on the generated risk predictions for the second portion of the plurality of human subjects.

2. The method of claim 1, wherein the first dataset is a training dataset and the second dataset is a validation dataset.

3. The method of claim 1, further comprising:
creating a third dataset corresponding to a third portion of the plurality of human subjects; and
providing the third dataset to the ML model with the at least one adjusted parameter to generate a risk prediction for developing the at least one disorder by the end of the target prediction period for each human subject included in the third portion of the plurality of human subjects.

4. The method of claim 1, further comprising:
determining whether each human subject of the plurality of human subjects has developed the at least one disorder by the end of the target prediction period;
labeling a portion of the plurality of human subjects who have developed the at least one disorder by the end of the target prediction period as positive for the disorder; and
labeling a remaining portion of the plurality of human subjects as healthy.

5. The method of claim 4, wherein determining that a human subject has developed the at least one disorder includes detecting at least one identifying factor in a final year of the target prediction period.

6. The method of claim 4, wherein the first portion of the plurality of human subjects has a first ratio of positive to healthy human subjects and the second portion of the plurality of human subjects has a second ratio of positive to healthy human subjects.

7. The method of claim 6, wherein the first ratio and the second ratio are different.

8. The method of claim 1, wherein selecting the at least one risk factor associated with developing the at least one disorder includes identifying at least one risk factor in a first year of the target prediction period associated with a diagnosis of the at least one disorder by the end of the target time period.

9. The method of claim 1, wherein the at least one risk factor corresponds to at least one Clinical Classifications Software Refined (CCSR) category.

10. The method of claim 1, wherein the at least one disorder is breast cancer.

11. The method of claim 1, wherein the trained ML model is configured to receive input data corresponding to a user and provide a risk prediction indicating the user's risk of being diagnosed with the at least one disorder by the end of the target prediction period.

12. The method of claim 11, wherein the risk prediction includes a risk score.

13. A system for training a machine learning (ML) model to assess the risk of a human subject for developing at least one disorder, comprising:
one or more computer systems programmed to perform operations comprising:
receiving an input dataset including at least medical claim data corresponding to a plurality of human subjects over a target prediction period;
splitting the input dataset into a first dataset corresponding to a first portion of the plurality of human subjects and a second dataset corresponding to a second portion of the plurality of human subjects;
selecting at least one risk factor associated with developing the at least one disorder from the first dataset;
training a machine learning (ML) model using the first dataset and the at least one risk factor, the ML model including at least one logistic regression model;
providing the second dataset to the ML model to generate a risk prediction for developing the at least one disorder by the end of the target prediction period for each human subject included in the second portion of the plurality of human subjects; and
tuning at least one parameter of the ML model based on the generated risk predictions for the second portion of the plurality of human subjects.

14. The system of claim 13, wherein the one or more computer systems is programmed to perform operations comprising:
creating a third dataset corresponding to a third portion of the plurality of human subjects; and
providing the third dataset to the ML model with the at least one adjusted parameter to generate a risk prediction for developing the at least one disorder by the end of the target prediction period for each human subject included in the third portion of the plurality of human subjects.

15. The system of claim 13, wherein the one or more computer systems is programmed to perform operations comprising:
determining whether each human subject of the plurality of human subjects has developed the at least one disorder by the end of the target prediction period;
labeling a portion of the plurality of human subjects who have developed the at least one disorder by the end of the target prediction period as positive for the disorder; and
labeling a remaining portion of the plurality of human subjects as healthy.

16. The system of claim 15, wherein determining that a human subject has developed the at least one disorder includes detecting at least one identifying factor in a final year of the target prediction period.

17. The system of claim 15, wherein the first portion of the plurality of human subjects has a first ratio of positive to healthy human subjects and the second portion of the plurality of human subjects has a second ratio of positive to healthy human subjects.

18. The system of claim 13, wherein selecting the at least one risk factor associated with developing the at least one disorder includes identifying at least one risk factor in a first year of the target prediction period associated with a diagnosis of the at least one disorder by the end of the target prediction period.

19. The system of claim 13, wherein the at least one risk factor corresponds to at least one Clinical Classifications Software Refined (CCSR) category.

20. The system of claim 13, wherein the at least one disorder is breast cancer.

21. The system of claim 13, wherein the trained ML model is configured to receive input data corresponding to a user and provide a risk prediction indicating the user's risk of being diagnosed with the at least one disorder by the end of the target prediction period.

22. The system of claim 21, wherein the risk prediction includes a risk score.
